(19) United States
(12) Patent Application Publication
(10) Pub. No.: US 2020/0354796 A1
(43) Pub. Date: Nov. 12, 2020

(54) Use of DNA Recombination Deficiency Score (RDS) in Cancer Treatment

(71) Applicant: SHUWEN BIOTECH CO., LTD., Huzhou City, Zhejiang (CN)

(72) Inventors: XINGMIN LI, Huzhou City, Zhejiang (CN); Lun PAN, Huzhou City, Zhejiang (CN); Can XIA, Huzhou City, Zhejiang (CN)

(21) Appl. No.: 16/652,080

(22) PCT Filed: Sep. 30, 2018
(86) PCT No.: PCT/CN2018/109006

§ 371 Date: Mar. 30, 2020

(30) Foreign Application Priority Data
Sep. 30, 2017 (CN) 201710927451.3

(51) Int. Cl. C12Q 1/6886

(52) U.S. Cl. CPC C12Q 1/6886; C12Q 2600/158; C12Q 2600/106

(57) ABSTRACT

The present invention provides a method for treating human cancer, which comprises predicting the sensitivity of tumor cells or tissues in a cancer patient to DNA damage therapy; and administering DNA damage therapy to the cancer patient. The invention also relates to kits to perform the method.

### RELATED APPLICATIONS AND PRIORITY

This application is a national phase entry of PCT/CN2018/109006 filed on Sep. 30, 2018 claiming the benefit of priority of the Chinese Application No. 201710927451.3 filed on Sep. 30, 2017, the text of both of which are incorporated herein in their entirety.

### FIELD OF THE INVENTION

The present invention generally relates to cancer treatment, particularly to a method for treating human cancer, comprising predicting the sensitivity of tumor cells or tissues in a cancer patient to DNA damage therapy; and administering DNA damage therapy to the cancer patient. The invention also relates to a kit to perform the method.

### BACKGROUND OF THE INVENTION

Homologous recombination (HR) and non-homologous end joining (NHEJ) are competing pathways that repair double-strand DNA breaks (DSBs) generated by certain cancer treatment modalities. HR also serves additional functions such as promoting cellular tolerance to DNA-damaging drugs that disrupt replication forks (Thompson, et al., 2001). Both HR and NHEJ facilitate DNA repair following the recruitment of upstream sensor/effector proteins. The HR pathway catalyzes DSB repair by identifying a stretch of homologous DNA and by replicating from this homologous DNA template, whereas NHEJ repairs DSBs by processing and religating the DSB ends.

When faced with a DSB, the cell's decision of whether to use HR or NHEJ is influenced by the cell cycle stage. NHEJ is the dominant pathway for repairing DSBs during the G₀-G₁ stages of the cell cycle, whereas HR occurs generally during S and G2. This regulation of repair is governed primarily by BRCA1 and 53BP1 proteins, which compete for occupancy at the DSB site. Stabilization of 53BP1 in cooperation with RIF1 leads to the exclusion of BRCA1 protein from the repair complex, and the DSB subsequently progresses to repair by NHEJ. If 53BP1 is excluded from the repair complex, then the DSB progresses to repair by HR. In this case, the DSB ends are processed into HR substrates, which involve 5′ to 3′ nuclease activity that generates 3′ single-stranded DNA (ssDNA) tails. This end processing is promoted by several proteins including CtP, BRCA1, and the MRN (Mre11/RAD50/NBS1) complex. The nuclease activity is also specifically triggered by interactions between Mre11 and cyclin-dependent kinase2, thereby promoting the phosphorylation of CtIP preferentially in S-G₂ cells. Similarly, mutations can arise if replication-disrupting lesions are not properly repaired before DNA replication, in which case these lesions may prompt homology-mediated polymerase template switching (Malkova, et al., 2012).

The efficiencies of these repair processes have important implications for carcinogenesis and malignant tumor progression. Like HR, the canonical version of NHEJ is thought to repair DNA with high fidelity (Arlt, et al., 2012; Guirouilh-Barbat, et al., 2004). However, some DSBs can undergo extensive degradation before religation using processes termed microhomology-mediated end joining and single-strand annealing, both of which create mutagenic deletions (Guirouilh-Barbat, et al., 2004; Bennardo, et al., 2008).

The cellular efficiencies of these repair processes can also directly affect tumor responsiveness during the treatment of cancer patients. The most marked examples are the hypersensitivities of HR-deficient tumors to poly(adenosinediphosphate-ribose) polymerase (PARP) inhibitors (Bryant, et al., 2004; Farmer, et al., 2004; O' Shaughnessy, et al., 2011) or platinum-based chemotherapies (Willers, et al., 2009; Birkelbach, et al., 2013). At present, methods to measure HR or NHEJ proficiency from human tumor biopsy tissues are limited (16,17). Some studies have measured the rate of DSB rejoining in tumors (such as H2AX phosphorylation kinetics), and rapid DSB rejoining may predict resistance of human tumors to radiotherapy and some chemotherapy drugs [reviewed in Redon, et al., 2012]. However, a single method that could successfully predict the relative efficiencies of HR and NHEJ is needed.

Thus, tumors that harbor ineffective error-free DNA repair machinery are likely to exhibit greater genomic instability, which is expected to drive malignant progression and generate more aggressive tumor phenotypes. A method that predicts error-free repair proficiency from human tumor biopsy tissues might have broad applications in clinical oncology as a prognostic indicator because genetic instability may indicate a greater propensity for malignant phenotypes like metastagenicity. What is more, a method that successfully quantifies repair efficiency might have important applications in clinical oncology because it would predict sensitivity of tumors to specific classes of treatment.

Triple-negative breast cancer (TNBC) is a unique subtype of breast cancer, accounting for about 15-20% of all breast cancers. According to the guideline of American Society of Clinical Oncology (ASCO)/American Association of Pathologists (CAP), TNBC is currently defined as ER/PR immunohistochemical (IHC) detection as 0 and HER2 IHC 0-1 or FISH<2.0.

Early TNBC is more susceptible to distant metastasis than other subtypes, and 5-year survival is worse. The recurrence risk of TNBC peaks at 3 years and decreases thereafter. However, the recurrence risk of non-TNBC is lower within 3 years, and this risk is maintained after that. The reason for the early recurrence of TNBC is largely due to the existence of residual disease, that is, pathological complete response (pCR) cannot be achieved, and the prognosis of TNBC patients who reach pCR is comparable to that of other subtypes of breast cancer, so it is urgent to seek effective treatment to increase pCR and improve prognosis.

Existing TNBC neoadjuvant treatment schemes are similar to non-TNBC, including anthracyclines, taxanes, cyclophosphamide, etc. and combinations thereof. In the CALGB 40603 study, combined carboplatin was able to increase breast pCR rates (60% vs 44%, P=0.0018) and breast/axillary pCR rates (54% vs 41%, P=0.0029). This indicates that DNA-damaging drugs such as platinum may be a new option for neoadjuvant treatment of TNBC patients, but it is still necessary to select suitable patients.

### SUMMARY OF THE INVENTION

The inventors have surprisingly found that the recombination deficiency score (RDS) is related to the sensitivity to DNA damage therapy. The lower the RDS value, the more sensitive the cancer cells are to the DNA damage therapy. Conversely, the higher the RDS value, the less sensitive the cancer cells are to the DNA damage therapy.

Thus, one aspect of the present invention provides a method for treating human cancer, comprising predicting the sensitivity of tumor cells or tissues in a cancer patient to DNA damage therapy; and administering DNA damage therapy to the cancer patient, wherein the predicting the sensitivity of tumor cells or tissues in the cancer patient to DNA damage therapy comprises obtaining a DNA recombination deficiency score (RDS) value of the tumor cells or tissues, and the RDS value is calculated by determining the expression levels of one or more DNA repair-related genes.

In one embodiment of the present invention, the DNA damage therapy is selected from DNA damage chemotherapy and the DNA damage radiotherapy.

According to embodiments of the present invention, the DNA damage chemotherapy comprises administrating a therapeutically effective amount of a chemical agent.

In specific embodiments of the present invention, the chemical agent is selected from the group of platinum compounds, DNA cross-linking agents, topoisomerase inhibitors, and PARP inhibitors. In specific embodiments of the present invention, the platinum compound is cisplatin or carboplatin. In specific embodiments of the present invention, the DNA cross-linking agent is cisplatin. In specific embodiments of the present invention, the topoisomerase inhibitor is topotecan. In specific embodiments of the present invention, the PARP inhibitor is olaparib. In specific embodiments of the present invention, the DNA damage radiotherapy comprises applying a medically tolerable radiation.

In the present invention, the DNA repair-related genes comprise at least one homologous recombination (HR) gene or non-homologous end joining (NHEJ) gene.

In embodiments of the present invention, the DNA repair-related genes include at least one gene from the group of RAD51, XRCC5, RIF1, PARPBP, PARP1, BRCA1, c-Met and E2F1, e.g. 1, 2, 3, 4, 5, 6, 7 or 8 genes from the group RAD5, XRCC5, RIF1, PARPBP, PARP1, BRCA1, c-Met and E2F1, preferably 2, 3, 4 or 5 genes from the group of RAD51, XRCC5, RIF1, PARPBP, PARP1, BRCA1, c-Met and E2F1.

In specific embodiments of the present invention, the DNA repair related genes include at least one gene from the group of RAD51, XRCC5, RIF1, PARPBP, PARP1, BRCA1 and c-Met, e.g. 1, 2, 3, 4, 5 or 6 genes from the group of RAD51, XRCC5, RIF1, PARPBP, PARP1, BRCA1 and c-Met, preferably 2, 3, 4 or 5 genes from the group of RAD51, XRCC5, RIF1, PARPBP, PARP1, BRCA1 and c-Met.

In specific embodiments of the present invention, the DNA repair-related genes include RAD51. In specific embodiments of the present invention, the DNA repair related genes include XRCC5. In specific embodiments of the present invention, the DNA repair related genes include PARPBP. In specific embodiments of the present invention, the DNA repair related genes include PARP1. In specific embodiments of the present invention, the DNA repair related genes include BRCA1. In specific embodiments of the present invention, the DNA repair related genes include RAD51 and XRCC5. In specific embodiments of the present invention, the DNA repair related genes include XRCC5 and BRCA1. In specific embodiments of the present invention, the DNA repair related genes include RAD51, XRCC5 and PARPBP. In specific embodiments of the present invention, the DNA repair related genes include RAD51, XRCC5 and BRCA1. In specific embodiments of the present invention, the DNA repair related genes include RAD 51, XRCC5, RIF1 and PARPBP. In specific embodiments of the present invention, the DNA repair related genes include RAD51, XRCC5, PARP1 and BRCA1. In specific embodiments of the present invention, the DNA repair related genes include RAD51, XRCC5, PARPBP and BRCA1. In specific embodiments of the present invention, the DNA repair related genes include RAD51, XRCC5, PARPBP, PARP1 and BRCA1. In specific embodiments of the present invention, the DNA repair related genes include RAD5, XRCC5, PARP1, BRCA1 and c-Met.

In the present invention, the RDS value is calculated by the following steps:

(1) subtracting the average value of the expression levels of each DNA repair related gene in the population with the expression level of the gene, and dividing by the standard deviation of the expression level of the gene in the population to obtain the Z value of the gene; (2) repeating step (1) to obtain the Z values of all DNA repair related genes; (3) multiplying the Z value of each DNA repair related gene by its weight, then summing the Z values of all DNA repair related genes to get the RDS value.

In a specific embodiment of the present invention, all of the weights of the DNA repair related genes are 1. In a specific embodiment of the present invention, the weights of the DNA repair related genes are determined by a random forest model. In a specific embodiment of the present invention, the resulting RDS value is multiplied by −1.

In a specific embodiment of the present invention, the DNA repair related genes are RAD51, XRCC5 and BRCA1, the corresponding Z values are Z_{RAD51}, Z_{XRCC5}, and Z_{BRCA1}, respectively, and the corresponding weights are 1.2677725, −2.1358314, and 1.8680589, respectively, and the RDS value is calculated by the formula:

RDS=1.8680589×Z_{BRCA1}−2.1358314×Z_{XRCC5}+1.2677725×Z_{RAD51}.

In a specific embodiment of the present invention, the DNA repair related genes are RAD51, XRCC5 and BRCA1, the corresponding Z values are Z_{RAD51}, Z_{XRCC5}, and Z_{BRCA1}, respectively, and the corresponding weights are 1.0862116, −1.3606527, and 1.8680589, respectively, and the RDS value is calculated by the formula:

RDS=1.2744411λZ_{BRCA1}−1.3606527×Z_{XRCC5}+1.0862116×Z_{RAD51}.

In a specific embodiment of the present invention, the DNA repair related genes are RAD51, XRCC5, PARPBP, PARP1 and BRCA1, the corresponding Z values are Z_{RAD51}, Z_{XRCC5}, Z_{PARPBP}, Z_{PARP1} and Z_{BRCA1}, respectively, and the corresponding weights are −0.9410212, 1.9078423, 1.2744411, 0.5792162 and −1.4464863, respectively. The RDS value is calculated by the formula:

RDS=−1×(1.9078423×Z_{XRCC5}−1.4464863×Z_{BRCA1}−0.9410212×Z_{RAD51}+0.9004490×Z_{PARPBP}+0.5792162×Z_{PARP1}).

In a specific embodiment of the present invention, the DNA repair related genes are RAD51, XRCC5, PARPBP, PARP1 and BRCA1, the corresponding Z values are Z_{RAD51}, Z_{XRCC5}, Z_{PARPBP}, Z_{PARP1} and Z_{BRCA1}, respectively, and the corresponding weights are −0.8562682, 1.8206667, 0.6713876, 0.5695937 and −1.2053798, respectively. The RDS value is calculated by the formula:

RDS=−1×(1.8206667×Z_{XRCC5}−1.20537983×Z_{BRCA1}−0.8562682×Z_{RAD51}+0.6713876×Z_{PARPBP}+0.5695937×Z_{PARP1}).

In a specific embodiment of the present invention, the DNA repair related genes are RAD51, XRCC5, PARPBP, PARP1 and BRCA1, the corresponding Z values are Z_{RAD51}, Z_{XRCC5}, Z_{PARPBP}, Z_{PARP1} and Z_{BRCA1}, respectively, and the corresponding weights are −0.8562682, 2.9992700, 1.8874888, 1.4543415 and −2.9277882, respectively. The RDS value is calculated by the formula:

RDS=−1×(2.9992700×Z_{XRCC5}−2.9277882×Z_{BRCA1}−0.8562682×Z_{RAD51}+1.8874888×Z_{PARPBP}+1.4543415×Z_{PARP1}).

In a specific embodiment of the present invention, the DNA repair related genes are RAD51, XRCC5, PARPBP, PARP1 and BRCA1, the corresponding Z values are Z_{RAD51}, Z_{XRCC5}, Z_{PARPBP}, Z_{PARP1} and Z_{BRCA1}, respectively, and the corresponding weights are −2.7960688, 3.4459578, 1.6877616, 1.7951636 and −3.1328142, respectively. The RDS value is calculated by the formula:

RDS=−1×(3.4459578×Z_{XRCC5}−3.1328142×Z_{BRCA1}−2.7960688×Z_{RAD51}+1.6877616×Z_{PARPBP}+1.7951636×Z_{PARP1}).

In a specific embodiment of the present invention, the DNA repair related genes are RAD51, XRCC5, PARPBP, PARP1 and BRCA1, the corresponding Z values are Z_{RAD51}, Z_{XRCC5}, Z_{PARPBP}, Z_{PARP1} and Z_{BRCA1}, respectively, and the corresponding weights are −1.5976511, 2.0106046, 1.1222873, 1.0772653 and −1.6125061, respectively. The RDS value is calculated by the formula:

RDS=−1×(2.0106046×Z_{XRCC5}−1.6125061×Z_{BRCA1}−1.5976511×Z_{RAD51}+1.1222873×Z_{PARPBP}+1.0772653×Z_{PARP1}).

In a specific embodiment of the present invention, the DNA repair related genes are RAD51, XRCC5, PARP1, BRCA1 and c-Met, the corresponding Z values are Z_{RAD51}, Z_{XRCC5}, Z_{PARP1}, Z_{BRCA1}, and Z_{c-Met}, respectively, and the corresponding weights are 1.5506891, −1.7869991, −1.3444708, 1.4939660 and 1.0868148, respectively. The RDS value is calculated by the formula:

RDS=−1×(1.4939660×Z_{BRCA1}−1.7869991×Z_{XRCC5}−1.3444708×Z_{PARP}+1.5506891×Z_{RAD51}+1.0868148×Z_{c-Met}).

In a specific embodiment of the present invention, the DNA repair related genes are RAD51, XRCC5, PARP1, BRCA1 and c-Met, the corresponding Z values are Z_{RAD51}, Z_{XRCC5}, Z_{PARP1}, Z_{BRCA1}, and Z_{c-Met}, respectively, and the corresponding weights are 1.8668920, −2.1714242, −1.6861369, 1.6586976 and 1.3319715, respectively, and the RDS value is:

RDS=−1×(1.6586976×Z_{BRCA1}−2.1714242×Z_{XRCC5}−1.6861369×Z_{PARP1}+1.8668920×Z_{RAD51}+1.3319715×Z_{c-Met}).

In a specific embodiment of the present invention, the DNA repair related genes are RAD51, XRCC5, PARP1, BRCA1 and c-Met, the corresponding Z values are Z_{RAD51}, Z_{XRCC5}, Z_{PARP1}, Z_{BRCA1}, and Z_{c-Met}, respectively, and the corresponding weights are 1.3497325, −2.1128981, −1.4465384, 1.9931659 and 1.2165381, respectively. The RDS value is calculated by the formula:

RDS=−1×(1.9931659×Z_{BRCA1}−2.1128981×Z_{XRCC5}−1.4465384×Z_{PARP1}+1.3497325×Z_{RAD51}+1.2165381×Z_{c-Met}).

In a specific embodiment of the present invention, the DNA repair related genes are RAD51, XRCC5, PARP1, BRCA1 and c-Met, respectively, the corresponding Z values are Z_{RAD51}, Z_{XRCC5}, Z_{PARP1}, Z_{BRCA1}, and Z_{c-Met}, respectively, and the corresponding weights are 1.3010898, −1.6731665, −1.1388590, 1.4318830 and 1.0790527, respectively. The RDS value is:

RDS=−1×(1.4318830×Z_{BRCA1}−1.6731665×Z_{XRCC5}−1.1388590×Z_{PARP1}+1.3010898×Z_{RAD51}+1.0790527×Z_{c-Met}).

In embodiments of the present invention, the expression level of the DNA repair related gene is the relative expression level to the expression level of one or more reference genes.

In specific embodiments of the present invention, the relative expression level is obtained by subtracting the expression level of the DNA repair related gene with the expression level of the reference genes.

In other embodiments of the present invention, the expression level of reference genes is the average value of the expression levels of all reference genes.

In embodiments of the present invention, the reference gene is at least one gene from the group of CALM2, B2M, TBP and GUSB.

In preferred embodiments of the invention, the cancer is selected from the group of pancreatic cancer, breast cancer, non-small cell lung adenocarcinoma, non-small cell lung cancer, colon cancer, lung cancer, non-small cell lung squamous cell carcinoma, esophageal cancer, and prostate cancer.

In other preferred embodiments of the invention, if the RDS value is lower than a preset cutoff value or falls within a preset interval, a DNA damage therapy is administered to the cancer patient.

In the present invention, the preset cutoff value or preset interval is obtained from samples of a population, specifically, (1) identifying N patients with cancer; (2) determining the sensitivity of tumor cells or tissues in a cancer patient to specific DNA damage therapies, and the most sensitive m % samples are considered sensitive samples; (3) obtaining the RDS values of tumor cells or tissues after sensitivity determining, the highest value or average value or median value or other distinguishing value of the RDS values in sensitive samples are used as preset cut-off value, the n % confidence interval is used as preset interval.

In embodiments of the present invention, the N is at least 20, 30, 50, 100 or larger. In embodiments of the present invention, the m is 1 to 50. In embodiments of the present invention, the m is selected from 10, 15, 25, 30, 40 and 50. In embodiments of the present invention, the n is 80 to 99. In embodiments of the present invention, the n is 95.

In embodiments of the present invention, the expression levels are obtained by nucleic acid hybridization/amplification. In embodiments of the present invention, the expression levels are obtained by FISH or CISH or RNA sequencing or DNA microarray. In embodiments of the present invention, the expression levels are obtained by qPCR. In embodiments of the present invention, the RDS value is obtained before or after administering the DNA damage therapy.

In embodiments of the present invention, the expression levels of the DNA repair related genes are the protein levels expressed by the DNA repair related genes. In embodiments of the present invention, the expression levels are obtained by IHC or ELISA or Western blot or protein microarray.

Another aspect of the present invention provides a diagnostic kit, comprising: primers for amplifying transcripts of the DNA repair related genes or probes that could hybridize with transcripts of the DNA repair related genes, or antibodies that could selectively immunoreact with proteins expressed by the DNA repair related genes.

In embodiments of the present invention, the DNA repair related genes comprise at least one gene from the group of RAD51, XRCC5, RIF1, PARPBP, PARP1, BRCA1, c-Met and E2F.

In specific embodiments of the present invention, for amplifying the transcript of RAD51 gene, the upstream primer is selected from the group of the sequences shown in SEQ ID NO. 1, SEQ ID NO. 4, SEQ ID NO. 7, SEQ ID NO. 10, and SEQ ID NO. 13; the downstream primer is selected from the group of sequences shown in SEQ ID NO. 2, SEQ ID NO. 5, SEQ ID NO. 8, SEQ ID NO. 11, and SEQ ID NO. 14. In specific embodiments of the present invention, for amplifying the transcript of XRCC5 gene, the upstream primer is selected from the group of sequences shown in SEQ ID NO. 16, SEQ ID NO. 19, SEQ ID NO. 22, SEQ ID NO. 35, and SEQ ID NO. 28; the downstream primer is selected from the group of sequences shown in SEQ ID NO. 12, SEQ ID NO. 20, SEQ ID NO. 23, SEQ ID NO. 26, and SEQ ID NO. 29. In specific embodiments of the present invention, for amplifying the transcript of the RIF1 gene, the upstream primer is selected from the group of the sequences shown in SEQ ID NO. 31, SEQ ID NO. 34, SEQ ID NO. 37 and SEQ ID NO. 40; the downstream primer is selected from the group of sequences shown in SEQ ID NO. 32, SEQ ID NO. 35, SEQ ID NO. 38 and SEQ ID NO. 41. In specific embodiments of the present invention, for amplifying the transcript of the PARPBP gene, the upstream primer is selected from the group of sequences shown in SEQ ID NO. 43, SEQ ID NO. 46, SEQ ID NO. 49 and SEQ ID NO. 52; the downstream primer is selected from the group of sequences shown in SEQ ID NO. 44, SEQ ID NO. 47, SEQ ID NO. 50 and SEQ ID NO. 53. In another embodiment of the present invention, the kit further comprises primers for amplifying the transcripts of one or more reference genes, or probes that could hybridize with transcripts of the reference genes, or antibodies that could selectively immunoreact with proteins expressed by the reference genes.

In another embodiment of the present invention, the reference gene is selected from the group of CALM2, B2M, TBP and GUSB.

In other specific embodiments of the present invention, the probe that could hybridize with the transcript of the RAD51 gene is selected from the group of sequences shown in SEQ ID NO. 3, SEQ ID NO. 6, SEQ ID NO. 9, SEQ ID NO. 12 and SEQ ID NO. 15. In other specific embodiments of the present invention, the probe that could hybridize with the transcript of the XRCC5 gene is selected from the group of sequences shown in SEQ ID NO. 18, SEQ ID NO. 21, SEQ ID NO. 24, SEQ ID NO. 26 and SEQ ID NO. 30. In other specific embodiments of the present invention, the probe that could hybridize with the transcript of the RIF1 gene is selected from the group of sequences shown in SEQ ID NO. 33, SEQ ID NO. 36, SEQ ID NO. 39 and SEQ ID NO. 42. In other specific embodiments of the present invention, the probe that could hybridize with the transcript of the PARPBP gene is selected from the group of sequences shown in SEQ ID NO. 45, SEQ ID NO. 48, SEQ ID NO. 51 and SEQ ID NO. 54.

In other specific embodiments of the present invention, the kit comprises antibodies that could selectively immunoreact with one or more of the proteins RAD51, XRCC5, RIF1, PARPBP, PARP1, BRCA1, C-MET and E2F1.

In other embodiments of the present invention, the kit further comprises primers for amplifying the transcripts of reference genes or probes that could hybridize with transcripts of reference genes, or antibodies that could selectively immunoreact with proteins expressed by the reference genes.

In other embodiments of the present invention, the reference gene is selected from the group of CALM2, B2M, TBP and GUSB.

In another specific embodiment of the present invention, for amplifying the transcripts of the CALM2 gene, the upstream primer is selected from the group of sequences shown in SEQ ID NO. 55, SEQ ID NO. 58 and SEQ ID NO. 61; the downstream primer is selected from the group of sequences shown in SEQ ID NO. 56, SEQ ID NO. 59 and SEQ ID NO. 62. In another specific embodiment of the present invention, for amplifying the transcript of the B2M gene, the upstream primer is selected from the group of sequences shown in SEQ ID NO. 64, SEQ ID NO. 67 and SEQ ID NO. 70; the downstream primer is selected from the group of sequences shown in SEQ ID NO. 65, SEQ ID NO. 68 and SEQ ID NO. 71. In another specific embodiment of the present invention, for amplifying the transcript of the TBP gene, the upstream primer is selected from the group of sequences shown in SEQ ID NO. 73, SEQ ID NO. 76, SEQ ID NO. 79 and SEQ ID NO. 82; the downstream primer is selected from the group of sequences shown in SEQ ID NO. 74, SEQ ID NO. 77, SEQ ID NO. 80 and SEQ ID NO. 83. In another specific embodiment of the present invention, for amplifying the transcript of the GUSB gene, the upstream primer is selected from the group of sequences shown in SEQ ID NO. 85, SEQ ID NO. 88 and SEQ ID NO. 91; the downstream primer is selected from the group of sequences shown in SEQ ID NO. 86, SEQ ID NO. 89 and SEQ ID NO. 92. In other embodiments of the present invention, the kit further comprises probes that could hybridize with the transcript of one or more of the genes CALM2, B2M, TBP and GUSB.

In another specific embodiment of the present invention, the probe that could hybridize with the transcript of the CALM2 gene is selected from the group of sequences shown in SEQ ID NO. 57, SEQ ID NO. 60 and SEQ ID NO. 63. In another specific embodiment of the present invention, the probe that could hybridize with the transcript of B2M gene is selected from the group of sequences shown in SEQ ID NO. 66, SEQ ID NO. 69 and SEQ ID NO. 72. In another specific embodiment of the present invention, the probe that could hybridize with the transcript of TBP gene is selected from the group of sequences shown in SEQ ID NO. 75, SEQ ID NO. 78, SEQ ID NO. 81 and SEQ ID NO. 84. In another specific embodiment of the present invention, the probe that could hybridize with the transcript of GUSB gene is selected from the group of sequences shown in SEQ ID NO. 87, SEQ ID NO. 90 and SEQ ID NO. 93.

In other specific embodiments of the present invention, the kit further comprises antibodies that could selectively immunoreact with one or more proteins expressed by CALM2, B2M, TBP and GUSB.

In other embodiments of the present invention, the probe is bound to a solid support.

In the embodiments above, the primers or probes can be labeled with any suitable detection label, including but not limited to radioisotope, fluorescein, biotin, enzyme (such as alkaline phosphatase), enzyme substrate, ligand, or antibody.

Throughout this application, the term “about” is used to indicate that a value includes the inherent variation of error for the measurement or quantitation method.

The use of the word “a” or “an” when used in conjunction with the term “comprising” may mean “one”, but it is also consistent with the meaning of “one or more”, “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this application in this specification can be implemented with respect to any method or composition of the invention, and vice versa. Furthermore, compositions of the invention can be used to achieve methods of the invention.

Other objects, features and advantages of the present invention will become apparent from the following detailed description.

### BRIEF DESCRIPTION OF THE DRAWINGS

FIG. 1 shows the relationship between RDS value and the sensitivities of different cell lines to different drugs.

FIGS. 2A-H shows box plots of expression levels of candidate genes at different P-M grades. A: z-G1 (RAD51), B: z-G2 (XRCC5), C: z-G3 (RIF1), D: z-G4 (PARPBP); E: z-G5 (PARP1); F: z-G6 (BRCA1); G: z-G10 (c-Met); H: z-G11 (E2F1).

FIG. 3 shows the ROC curve of RDS₁ regression for predicting the risk of pCR-1. The area under the curve (AUC) is 0.782.

FIG. 4 shows the ROC curve of RDS₂ regression for predicting the risk of pCR-1. The area under the curve (AUC) is 0.787.

FIG. 5 shows the ROC curve of RDS₃ regression for predicting the risk of pCR-1. The area under the curve (AUC) is 0.788.

FIG. 6 shows the ROC curve of RDS₄ regression for predicting the risk of pCR-1. The area under the curve (AUC) is 0.800.

FIG. 7 shows the ROC curve of RDS₅ regression for predicting the risk of pCR-1. The area under the curve (AUC) is 0.788.

FIG. 8 shows the ROC curve of RDS₆ regression for predicting the risk of pCR-1. The area under the curve (AUC) is 0.814.

FIG. 9 shows the ROC curve of RDS₇ regression for predicting the risk of pCR-1. The area under the curve (AUC) is 0.813.

FIG. 10 shows the ROC curve of RDS₈ regression for predicting the risk of pCR-1. The area under the curve (AUC) is 0.780.

FIG. 11 shows the ROC curve of RDS₉ regression for predicting the risk of pCR-1. The area under the curve (AUC) is 0.778.

FIG. 12 shows the ROC curve of RDS₁₀ regression for predicting the risk of pCR-1. The area under the curve (AUC) is 0.779.

FIG. 13 shows the ROC curve of RDS₁₁ regression for predicting the risk of pCR-1. The area under the curve (AUC) is 0.779.

### DETAILED DESCRIPTION OF THE INVENTION

To date, there is still a lack of suitable “pharmaceutically acceptable” targets in cancer and effective targeted therapies for a particular subtype of cancer. By establishing a DNA recombination deficiency score (RDS) system, the inventors have surprised found that some cancer cells had lower RDS values, while others had higher RDS values. The inventors have further surprisingly found that cells with lower RDS values are more sensitive to DNA damage therapy, while cells with higher RDS values are less sensitive to DNA damage therapy.

1. RDS

In some embodiments of the present invention, the RDS value can be obtained by the expression levels of the DNA repair related genes.

In specific embodiments of the present invention, the RDS value is calculated by the following steps:

(1) subtracting the average value of the expression levels of each DNA repair related gene in the population with the expression level of the gene, and dividing by the standard deviation of the expression level of the gene in the population to obtain the Z value of the gene; (2) repeating step (1) to obtain the Z values of all DNA repair related genes; (3) multiplying the Z value of each DNA repair related gene by its weight, then summing the Z values of all DNA repair related genes to get the RDS value.

In a specific embodiment of the present invention, all of the weights of the DNA repair related genes are 1. In a specific embodiment of the present invention, the weights of the DNA repair related genes are determined by a random forest model. In a specific embodiment of the present invention, the resulting RDS value is multiplied by −1.

In some embodiments of the present invention, the DNA repair related genes comprise at least one homologous recombination (HR) gene or non-homologous end joining (NHEJ) gene.

In some embodiments of the present invention, the DNA repair related genes comprise at least 1 gene selected from RAD51, XRCC5, RIF1, PARPBP, PARP1, BRCA1, c-Met and E2F1, e.g. 1, 2, 3, 4, 5, 6, 7 or 8 genes of RAD51, XRCC5, RIF1, PARPBP, PARP1, BRCA1, c-Met and E2F1, preferably 2, 3, 4 or 5 genes of RAD51, XRCC5, RIF1, PARPBP, PARP1, BRCA1, c-Met and E2F1. In some embodiments of the present invention, the DNA repair related genes comprise at least 1 gene of RAD51, XRCC5, RIF1, PARPBP, PARP1, BRCA1 and c-Met, e.g. 1, 2, 3, 4, 5 or 6 genes of RAD51, XRCC5, RIF1, PARPBP, PARP1, BRCA1 and c-Met, preferably 2, 3, 4 or 5 genes of RAD51, XRCC5, RIF1, PARPBP, PARP1, BRCA1 and c-Met. In specific embodiments of the present invention, the DNA repair related genes comprise RAD51. In specific embodiments of the present invention, the DNA repair related genes comprise XRCC5. In specific embodiments of the present invention, the DNA repair related genes comprise PARPBP. In specific embodiments of the present invention, the DNA repair related genes comprise PARP1. In specific embodiments of the present invention, the DNA repair related genes comprise BRCA1. In specific embodiments of the present invention, the DNA repair related genes comprise RAD51 and XRCC5. In specific embodiments of the present invention, the DNA repair related genes comprise XRCC5 and BRCA1. In specific embodiments of the present invention, the DNA repair related genes comprise RAD51, XRCC5 and PARPBP. In specific embodiments of the present invention, the DNA repair related genes comprise RAD51, XRCC5 and BRCA1. In specific embodiments of the present invention, the DNA repair related genes comprise RAD 51, XRCC5, RIF1 and PARPBP. In specific embodiments of the present invention, the DNA repair related genes comprise RAD51, XRCC5, PARP1 and BRCA1. In specific embodiments of the present invention, the DNA repair related genes comprise RAD51, XRCC5, PARPBP and BRCA1. In specific embodiments of the present invention, the DNA repair related genes comprise RAD51, XRCC5, PARPBP, PARP1 and BRCA1. In specific embodiments of the present invention, the DNA repair related genes comprise RAD51, XRCC5, PARP1, BRCA1 AND c-Met.

In embodiments of the present invention, the expression level of the DNA repair related gene is the relative expression level to the expression level of reference genes. In specific embodiments of the present invention, the relative expression level is obtained by subtracting the expression level of the DNA repair related gene with the expression level of the reference genes. In other embodiments of the present invention, the expression level of reference genes is the average value of the expression levels of all reference genes.

In embodiments of the present invention, the reference gene is selected from at least one of CALM2, B2M, TBP and GUSB.

Various techniques suitable for detecting gene expression levels in cell samples are known, such as fluorescence in situ hybridization (FISH), chromogenic in situ hybridization (CISH), and real-time PCR.

In embodiments of the present invention, gene expression is detected using real-time quantitative PCR or qPCR. The target DNA to be determined can be amplified by real-time PCR using, for example, conventional techniques such as TaqMan, Scorpion, and molecular markers, and the amount of the amplified DNA product can be detected by a non-sequence-specific fluorescent dye (for example, SybrGreen), or a labeled probe such as TaqMan probe, FRET probe or a molecular marker. For expression level analysis, as known in the art, an endogenous housekeeping gene can be used as a reference. Quantitative real-time PCR is particularly suitable for determining mRNA levels of genes in cell or tissue samples. In this case, the mRNA is first reverse transcribed into cDNA and then amplified by PCR using specific oligonucleotide PCR primers. The qRT-PCR method is well known in the art.

To detect the protein expression of a gene in cancer cells or tissue samples, any known method for measuring the protein level in cells or tissue samples can be used in the present invention. Examples of these methods include, but are not limited to, immunohistochemistry (IHC), ELISA, western blot, protein microarray, and the like. Generally, an antibody that specifically immunoreacts with a protein is contacted with cells or tissue samples under conditions of an immune reaction with a gene, and the amount of protein and bound antibody in the sample is measured. In IHC analysis, FFPE tumor samples can often be used. For ELISA, western blot, and protein microarray analysis, the sample can be a FFPE sample or a fresh frozen sample, and is preferably homogenized and extracted prior to contact with the antibody, as generally known in the art.

In preferred embodiments, RDS value of cancer cells or tissues in a patient is determined by in situ hybridization (FISH) analysis or real-time quantitative PCR. In other preferred embodiments, the RDS value of cancer cells or tissues in a patient is determined by qRT-PCR.

In one aspect of the present invention, RDS can provide a method for treating human cancer, comprising: predicting the sensitivity of tumor cells or tissues in a cancer patient to DNA damage therapy; and administering DNA damage therapy to the cancer patient, wherein the predicting the sensitivity of tumor cells or tissues in a cancer patient to DNA damage therapy refers to obtaining a DNA recombination deficiency score (RDS) of the tumor cells or tissues. The magnitude of the RDS value can be used to guide the treatment of cancer patients. The magnitude of the RDS value can be used to guide the treatment of cancer patients. Specifically, only if the RDS value is lower than a preset cutoff value or falls within a preset interval, a therapeutically effective amount of a DNA damage drug or a DNA damage therapy is administered to the cancer patient.

In a preferred embodiment of the present invention, the method of treating human cancer comprises identifying cancer patient with or diagnosed with cancer; obtaining RDS value of tumor cells or tissues in the cancer patient; and administering DNA damage therapy to the cancer patient.

It is known in the art that DNA damage therapy is selected from at least one of a DNA damage chemotherapy or a DNA damage radiotherapy.

According to the embodiments of the present invention, the DNA damage therapy is selected from DNA damage chemotherapy or the DNA damage radiotherapy. In specific embodiments of the present invention, the chemotherapeutic agent may be a platinum-based compound, such as cisplatin, carboplatin, oxaliplatin, satraplatin, picoplatin, Nedaplatin, Triplatin, Lipoplatin, or a liposomal version of cisplatin.

In specific embodiments, the chemotherapeutic agent may be a DNA cross-linker. Alkylating agents such as 1, 3-bis(2-chloroethyl)-1-nitrosourea (BCNU, carmustine)) and nitrogen mustard which are used in chemotherapy can cross link with DNA at N7 position of guanine on the opposite strands forming interstrand crosslink. Cisplatin (cis-diaminedichloroplatinum(II)) and its derivatives forms DNA cross links as monoadduct, interstrand crosslink, intrastrand crosslink or DNA protein crosslink. Mostly it acts on the adjacent N-7 guanine forming 1, 2 intrastrand crosslink.

In further embodiments, the chemotherapeutic agent may be a topoisomerase inhibitor. Topoisomerase inhibitors are drugs that affect the activity of two enzymes: topoisomerase I and topoisomerase II. When the DNA double-strand helix is unwound, during DNA replication or transcription, for example, the adjacent unopened DNA winds tighter (supercoils), like opening the middle of a twisted rope. The stress caused by this effect is in part aided by the topoisomerase enzymes. They produce single- or double-strand breaks into DNA, reducing the tension in the DNA strand. This allows the normal unwinding of DNA to occur during replication or transcription. Inhibition of topoisomerase I or II interferes with both of these processes. Two topoisomerase I inhibitors, irinotecan and topotecan, are semi-synthetically derived from camptothecin, which is obtained from the Chinese ornamental tree Camptotheca acuminate. Drugs that target topoisomerase II can be divided into two groups. The topoisomerase II poisons cause increased levels enzymes bound to DNA. This prevents DNA replication and transcription, causes DNA strand breaks, and leads to programmed cell death (apoptosis). These agents include etoposide, doxorubicin, mitoxantrone and teniposide. The second group, catalytic inhibitors, are drugs that block the activity of topoisomerase II, and therefore prevent DNA synthesis and translation because the DNA cannot unwind properly. This group includes novobiocin, merbarone, and aclarubicin, which also have other significant mechanisms of action.

In still further embodiments, the chemotherapeutic agent may be a PARP inhibitor. As used herein, “PARP inhibitor” (i.e., an inhibitor of poly ADP ribose polymerase) shall mean an agent that inhibits PARP more than it inhibits any other polymerase. In one embodiment, the PARP inhibitor inhibits PARP at least two-fold more than it inhibits any other polymerase. In another embodiment, the PARP inhibitor inhibits PARP at least 10-fold more than it inhibits any other polymerase. In a third embodiment, the PARP inhibitor inhibits PARP more than it inhibits any other enzyme. In one particular embodiment, the PARP inhibitor is olaparib, rucaparib, veliparib, CEP 9722, MK 4827, BMN-673, 3-aminobenzamide, a tetracycline compound, 4-hydroxyquinazoline and a derivative thereof, and a carboxamino-benzimidazole and a derivative thereof.

In some embodiments, the chemotherapeutic agent is any of (and in some embodiments selected from the group consisting of) alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, 111.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional chemotherapeutic agents include the cytotoxic agents useful as antibody drug conjugates, such as maytansinoids (DM1, for example) and the auristatins MMAE and MMAF, for example.

The method of the invention is applicable to all these chemotherapeutic agents.

In the present invention, cancer is also called malignant tumor, which refers to a new organism formed by the abnormal proliferation and differentiation of local tissue cells because the gene levels lose normal regulation of their growth due to various tumorigenic factors.

In preferred embodiments of the present invention, the cancer is selected from the group of pancreatic cancer, breast cancer, non-small cell lung adenocarcinoma, non-small cell lung cancer, colon cancer, lung cancer, non-small cell lung squamous cell carcinoma, esophageal cancer, and prostate cancer

In preferred embodiments of the present invention, the method of treating human cancer comprises identifying cancer patient with or diagnosed with cancer; obtaining RDS value of tumor cells or tissues in the cancer patient; and administering DNA damage therapy to the cancer patient if the RDS value is lower than a preset cutoff value or falls within a preset interval. In other words, the method comprises administering a DNA damage therapy to a patient diagnosed with cancer and whose RDS value is lower than a preset cutoff value or falls within a preset interval.

According to the embodiments of the present invention, the preset cutoff value or preset interval is obtained from samples of a population, specifically, (1) identifying N patients with cancer; (2) determining the sensitivity of tumor cells or tissues in cancer patients to specific DNA damage therapies, and the most sensitive m % samples are considered sensitive samples; (3) obtaining the RDS value of tumor cells or tissues after sensitivity determining, the highest value or average value or median value or other distinguishing value of the RDS values in sensitive samples are used as preset cut-off value, the n % confidence interval is used as preset interval. In certain embodiments of the present invention, the N is 22. In certain embodiments of the present invention, the m is 25.

3. Kits

The present invention further provides a kit for obtaining RDS value in cells from a patient. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting RDS values in cancer cells in accordance with the present invention using the above-discussed detection techniques.

The kit can further comprise reagents for labeling mRNA of genes to be measured in the sample. The kit may also include labeling reagents, including at least one of amine-modified nucleotide, poly(A) polymerase, and poly(A) polymerase buffer. Labeling reagents can include an amine-reactive dye.

In certain embodiments of the present invention, the kit comprises primers using for amplifying the transcripts of the genes RAD1, XRCC5, RIF1, PARPBP, PARP1, BRCA1, C-MET and E2F. In certain embodiments, the primers may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, or 300 nucleotides.

In the specific embodiments, for amplifying the transcript of RAD51 gene, the upstream primer is selected from sequences shown in SEQ ID NO. 1, SEQ ID NO. 4, SEQ ID NO. 7, SEQ ID NO. 10, and SEQ ID NO. 13; the downstream primer is selected from sequences shown in SEQ ID NO. 2, SEQ ID NO. 5, SEQ ID NO. 8, SEQ ID NO. 11, and SEQ ID NO. 14. For amplifying the transcript of XRCC5 gene, the upstream primer is selected from sequences shown in SEQ ID NO. 16, SEQ ID NO. 19, SEQ ID NO. 22, SEQ ID NO. 35, and SEQ ID NO. 28; the downstream primer is selected from sequences shown in SEQ ID NO. 12, SEQ ID NO. 20, SEQ ID NO. 23, SEQ ID NO. 26, and SEQ ID NO. 29. For amplifying the transcript of RIF1 gene, the upstream primer is selected from sequences shown in SEQ ID NO. 31, SEQ ID NO. 34, SEQ ID NO. 37 and SEQ ID NO. 40; the downstream primer is selected from sequences shown in SEQ ID NO. 32, SEQ ID NO. 35, SEQ ID NO. 38 and SEQ ID NO. 41. For amplifying the transcript of PARPBP gene, the upstream primer is selected from sequences shown in SEQ ID NO. 43, SEQ ID NO. 46, SEQ ID NO. 49 and SEQ ID NO. 52; the downstream primer is selected from sequences shown in SEQ ID NO. 44, SEQ ID NO. 47, SEQ ID NO. 50 and SEQ ID NO. 53.

In another embodiments, the kit comprises probe that could hybridize with the transcript of the genes RAD5, XRCC5, RIF1, PARPBP, PARP1, BRCA1, C-MET and E2F1.

In another embodiments, the probe that could hybridize with the transcript of RAD51 gene is selected from sequences shown in SEQ ID NO. 3, SEQ ID NO. 6, SEQ ID NO. 9, SEQ ID NO. 12 and SEQ ID NO. 15. The probe that could hybridize with the transcript of XRCC5 gene is selected from sequences shown in SEQ ID NO. 18, SEQ ID NO. 21, SEQ ID NO. 24, SEQ ID NO. 26 and SEQ ID NO. 30. The probe that could hybridize with the transcript of RIF1 gene is selected from sequences shown in SEQ ID NO. 33, SEQ ID NO. 36, SEQ ID NO. 39 and SEQ ID NO. 42. The probe that could hybridize with the transcript of PARPBP gene is selected from sequences shown in SEQ ID NO. 45, SEQ ID NO. 48, SEQ ID NO. 51 and SEQ ID NO. 54.

In other specific embodiments, the kit antibodies that could selectively immunoreact with proteins expressed by the genes RAD51, XRCC5, RIF1, PARPBP, PARP1, BRCA1, C-MET and E2F1.

In other embodiments, the kit further comprises primers for amplifying the transcripts of reference genes CALM2, B2M, TBP and GUSB.

In another specific embodiments, for amplifying the transcripts of CALM2 gene, the upstream primer is selected from sequences shown in SEQ ID NO. 55, SEQ ID NO. 58 and SEQ ID NO. 61; the downstream primer is selected from sequences shown in SEQ ID NO. 56, SEQ ID NO. 59 and SEQ ID NO. 62. For amplifying the transcript of B2M gene, the upstream primer is selected from sequences shown in SEQ ID NO. 64, SEQ ID NO. 67 and SEQ ID NO. 70; the downstream primer is selected from sequences shown in SEQ ID NO. 65, SEQ ID NO. 68 and SEQ ID NO. 71. For amplifying the transcript of TBP gene, the upstream primer is selected from sequences shown in SEQ ID NO. 73, SEQ ID NO. 76, SEQ ID NO. 79 and SEQ ID NO. 82; the downstream primer is selected from sequences shown in SEQ ID NO. 74, SEQ ID NO. 77, SEQ ID NO. 80 and SEQ ID NO. 83. For amplifying the transcript of GUSB gene, the upstream primer is selected from sequences shown in SEQ ID NO. 85, SEQ ID NO. 88 and SEQ ID NO. 91; the downstream primer is selected from sequences shown in SEQ ID NO. 86, SEQ ID NO. 89 and SEQ ID NO. 92.

In other embodiments, the kit further comprises probes that could hybridize with transcripts of reference genes CALM2, B2M, TBP and GUSB.

In other specific embodiments, the probe that could hybridize with the transcript of CALM2 gene is selected from sequences shown in SEQ ID NO. 57, SEQ ID NO. 60 and SEQ ID NO. 63. The probe that could hybridize with the transcript of B2M gene is selected from sequences shown in SEQ ID NO. 66, SEQ ID NO. 69 and SEQ ID NO. 72. The probe that could hybridize with the transcript of TBP gene is selected from sequences shown in SEQ ID NO. 75, SEQ ID NO. 78, SEQ ID NO. 81 and SEQ ID NO. 84. The probe that could hybridize with the transcript of GUSB gene is selected from sequences shown in SEQ ID NO. 87, SEQ ID NO. 90 and SEQ ID NO. 93.

In other specific embodiments, the kit comprises antibodies that could selectively immunoreact with proteins expressed by the reference genes CALM2, B2M, TBP and GUSB.

In other embodiments, the probe is bound to a solid support.

In the embodiments above, the primers or probes can be labeled with any suitable detection label, including but not limited to radioisotope, fluorescein, biotin, enzyme (such as alkaline phosphatase), enzyme substrate, ligand, or antibody.

Alternatively, the probes and primers included in the kit are not labeled, and instead, one or more markers are provided in the kit so that users may label the oligonucleotides at the time of use.

In still other embodiments, the kit may include antibodies that could selectively immunoreact with proteins expressed by the DNA repair related genes and useful in immunohistochemical analysis of protein expressions of DNA repair related genes in cell or tissue sample from a patient.

In addition, the detection kit preferably includes instructions on using the kit for obtaining the RDS value in cell or tissue from a patient, in accordance with the detailed description above.

Typically, once the RDS value is lower than a preset cutoff value or falls within a preset interval is determined in a lab, physicians or patients or other researchers may be informed of the result. Specifically the result may be cast in a transmittable form that can be communicated or transmitted to other researchers or physicians or genetic counselors or patients. Such a form can vary and can be tangible or intangible. The result with regard to the presence or absence of in the individual tested can be embodied in descriptive statements, diagrams, photographs, charts, images or any other visual forms. The statements and visual forms can be recorded on a tangible media such as papers, computer readable media such as floppy disks, compact disks, etc., or on an intangible media, e.g., an electronic media in the form of email or website on internet or intranet. In addition, the result may also be recorded in a sound form and transmitted through any suitable media, e.g., analog or digital cable lines, fiber optic cables, etc., via telephone, facsimile, wireless mobile phone, internet phone and the like. The test result may be received and/or input into a computer system and processed by a computer program product in the computer system, e.g., in a hospital or clinic.

### Benefit of the Present Invention

RDS can accurately predict the sensitivity of DNA damage therapy. It is related to the degree of genome instability in tumor cells and can provide valuable information that cannot be obtained by existing diagnostic methods. RDS is a new scoring system that predicts DSB repair pathway selection by quantifying the expression of four genes. In particular, the mRNA expression of DNA repair-related genes in cancer cell lines was compared with the sensitivity of DNA damage agents. This identified gene expression scoring system is called RDS, and it is inversely related to the level of DNA repair gene expression. Low RDS value can identify HR-deficient tumors while being hypersensitive to specific DNA damage therapies.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the claims.

### Example 1 C Pacer Cells' Sensitivity to DNA Damage Drugs

1. Regents

1640 medium (Gibco), DMEM medium (Hyclone), MEM medium (Gibco), F12-K medium (Gibco), DMEM/F12 medium (Gibco), L-15 medium (Hyclone), IMDM culture Base (Hyclone), non-essential amino acid (Gibco), sodium pyruvate (Gibco), insulin-transferrin-selenium additive TS-G, Shanghai Yuanpei Bio-tech), fetal bovine serum (seagreen), trypsin digestion fluid (Jiangsu KGI), Cell Titer 96® AQueousOne Solution Cell Proliferation Assay (Promega), 96-well cell culture plate (Corning, Cat. No. 3599), Cisplatin (sigma, Cat. No. P4394), Olaparib (Selleck, Cat. No. S1060), Topotecan hydrochloride hydrate (Sigma, article number T2705), Paclitaxel (Dalian Meilun, article number MB1178).

2. Cell Lines

| Cell lines | Cell types | Culture condition |
|---|---|---|
| Capan-1 | Human pancreatic cancer cells | IMEM + 10% FBS |
| HCC1937 | Human breast cancer cells | 1640 + 10% FBS |
| A549 | Human non-small cell lung | |
| | adenocarcinoma cells | |
| H1299 | Human non-small cell lung | |
| | cancer cells | |
| HT29 | Human colon cancer cells | |
| H1975 | Human non-small cell lung | |
| | adenocarcinoma cells | |
| H1650 | Human lung cancer cells | |
| H2228 | Human non-small cell lung | |
| | adenocarcinoma cells | |
| MCF-7 | Human breast cancer cells | |
| HCC95 | Human breast cancer cells | |
| H520 | Human non-small cell lung | |
| | squamous cell | |
| HCC827 | Human breast cancer cells | |
| HCC4006 | Human breast cancer cells | |
| H3122 | Human breast cancer cells | |
| KYSE450 | Human esophageal cancer cells | |
| H2444 | Human non-small cell lung | |
| | cancer cells | |
| CAMA-1 | Human breast cancer cells | MEM + 10% |
| | | FBS + NEAA + |
| | | Sodium pyruvate |
| HEK-293 | Human embryonic kidney cells | DMEM + 20% FBS |
| T84 | Human colon cancer cells | DMEM/F12 + 5% |
| | | FBS |
| PC-3 | Human prostate cancer cells | F-12K + 10% FBS |
| MDA-M | Human breast cancer cells | DMEM + 10% |
| B-436 | | FBS + 10 mcg/ml |
| | | ITS-G |
| Cal-120 | Human breast cancer cells | DMEM + 10% FBS |

### 3. In Vitro Drug Sensitivity Test of Cells—MTS Method

When the cell density in the culture flask reached 80-90%, the cells were collected. Then the inventors adjusted the cell suspension concentration to 1×10⁴/mL, added the cells to a 96 well plate as 100 ul per well, that is, 1000-2000 cells per well. Then the cells were incubated at 5% CO₂, 37° C. overnight. The next day, after the cells at the bottom of the 96-well plate adhered, various drugs with different concentration gradients were added: Olaparib (2.5 μM, 5 μM, 10 μM, 20 μM, 40 μM, 80 μM, 160 μM), Cisplatin (0.0625 μM, 0.125 μM, 0.25 μM, 0.5 μM, 1 μM, 2 μM, 4 μM, 8 μM, 16 μM), Topotecan (0.0078125 μM, 0.015625 μM, 0.3125 μM, 0.0625 μM, 0.125 μM, 0.25 μM, 0.5 μM, 1 μM), Paclitaxel (0.001953125 μM, 0.00390625 μM, 0.0078125 μM, 0.015625 μM, 0.3125 μM, 0.0625 μM, 0.125 μM). After putting the 96-well plate into a CO₂ incubator for 72 hours, the cell morphology and density in each were observed. For slow-growing cells, supplementation treatment was needed. For different cells, the culture time was between 72 h-216 h, and most of the cell culture time was between 144 h-168 h. When the preset culture time is reached, removed the 96-well plate, added 90 μL of culture medium and 10 μL CellTiter 96® AQueous One Solution Reagent to each well, and incubated in the CO₂ incubator for 1-4 hours, then placed the 96-well plate on the microplate reader. Measure the absorbance (OD) of each well at 492 nm. Cell Viabilities was calculated by the formula:

$$Cell Viability = \frac{Drug OD - Blank OD}{Negative OD - Blank OD}$$

4. Results

TABLE 1
The IC50s of 22 cell lines to different drugs

| | IC50 (μM) | | | |
|---|---|---|---|---|
| Cell lines | Cisplatin | Olaparib | Topotecan | Paclitaxel |
| Capan-1 | 0.2145 | 2.574 | 0.0158 | 0.006985 |
| HCC1937 | 0.2592 | 10 | 0.0352 | 0.009 |
| A549 | 1 | 80 | 0.25 | 0.005859 |
| H1299 | 2 | 40 | 0.0287 | 0.0227 |
| HT29 | 0.6915 | 7.66 | 0.015625 | 0.003054 |
| H1975 | 6.998 | 80 | 0.25 | 0.006998 |
| H1650 | 1.065 | 75 | 0.125 | 0.006609 |
| H2228 | 0.5145 | 10.8 | 0.02462 | 0.005201 |
| MCF-7 | 0.8935 | 3.51 | 0.002048 | 0.004841 |
| HCC95 | 0.8784 | 25.33 | 0.04672 | 0.006127 |
| H520 | 0.4642 | 2.192 | 0.009174 | 0.006993 |
| HCC827 | 0.6259 | 6.081 | 0.01257 | 0.00926 |
| HCC4006 | 1 | 9.14 | 0.0231 | 0.003852 |
| H3122 | 1.536 | 16.88 | 0.01999 | 0.008484 |
| KYSE450 | 1.462 | 129.8 | 0.02631 | 0.01146 |
| H2444 | 1.137 | 7.672 | 0.008085 | 0.0071 |
| CAMA-1 | 1.4 | 8.33 | 0.0009069 | 0.004164 |
| HEK-293 | 0.465 | 5.879 | 0.01796 | 0.01029 |
| T84 | 0.4329 | 5.85 | 0.00756 | 0.03209 |
| PC-3 | 0.7986 | 6.221 | 0.02009 | 0.004268 |
| MDA-MB-436 | 0.1393 | <2.5 | 0.004211 | 0.009669 |
| Cal-120 | 4.437 | 160 | 0.5176 | 0.04345 |

The results show that for any DNA damaging drug, such as cisplatin, olaparib, or topotecan, different cell lines show different IC50s, indicating that their sensitivity to the same DNA damaging drug is different. For the non DNA damage drug paclitaxel, the sensitivity of different cell lines showed little difference.

For the first drug experiment, 25% samples (5) with the highest sensitivities (ie, the lowest IC50) were selected as the sensitive samples, and 25% samples (5) with the lowest sensitivities (i.e. the highest IC50) were selected as the non-sensitive samples, details as follows:

TABLE 2
Sensitive cell lines and non-sensitive cell lines for each drug in the test

| Cisplatin | | Olaparib | | Topotecan | | Paclitaxel | |
|---|---|---|---|---|---|---|---|
| | Non- | | Non- | | Non- | | Non- |
| Sensitive | sensitive | Sensitive | sensitive | Sensitive | sensitive | Sensitive | sensitive |
| MDA-MB-436 | KYSE450 | H520 | H1650 | CAMA-1 | HCC95 | HT29 | HEK-293 |
| Capan-1 | H3122 | MDA-MB-436 | A549 | MCF-7 | H1650 | HCC4006 | KYSE450 |
| HCC1937 | H1299 | Capan-1 | H1975 | MDA-MB-436 | A549 | CAMA-1 | H1299 |
| T84 | Cal-120 | MCF-7 | KYSE450 | T84 | H1975 | PC-3 | T84 |
| H520 | H1975 | T84 | Cal-120 | H2444 | Cal-120 | MCF-7 | Cal-120 |

### Example 2 Determine RDS in Cell Lines

In order to obtain the RDS value of each cell line, eight DNA repair related genes were selected, namely RAD51, XRCC5, RIF1, PARPBP, PARP1, BRCA1, C-MET and E2F1. In order to obtain the relative expression levels of these eight genes, four reference genes were selected, namely CALM2, B2M, TBP and GUSB.

1. Materials

AxyPrep Total RNA Miniprep Kit (axygen, Cat. No.: AP-MN-MS-RNA-50G); cell lines.

TABLE 3
The parameters of cell lines used for sensitivity testing

| Cell Line | Date | CONC (ng/ul) | Cells (10E6) |
|---|---|---|---|
| HCC1937 | 160803 | 455 | 2.564 |
| H358 | 160819 | 355 | 10 |
| HCC1937 | 160822 | 455 | 3.37 |
| CAPON1 | 160829 | 478 | 4.264 |
| H1299 | 160913 | 650 | 7.12 |
| A549 | 160913 | 482 | 6.8 |
| HT29 | 160913 | 877 | 9.78 |
| H1975 | 160923 | 325 | 2.912 |
| H1650 | 161013 | 640 | 2.668 |
| MCF-7 | 161013 | 690 | 7.92 |
| H3122 | 161017 | 475 | 5.4 |
| HCC95 | 161017 | 370 | 7.02 |
| H2228 | 161024 | 680 | 8.685 |
| H520 | 161024 | 660 | 9.768 |
| HCC827 | 161024 | 500 | 2.375 |
| KYSE450 | 161024 | 280 | 10.8 |
| H2444 | 161031 | 770 | 3.52 |
| H4006 | 161031 | 640 | 3.126 |
| CAL-120 | 161103 | 480 | 4.488 |
| KYSE450 | 161107 | 425 | 8 |
| HCC827 | 161110 | 755 | 6.02 |
| H2444 | 161114 | 827 | 5.135 |
| MDA-MB-436 | 161117 | 820 | 7 |
| T84 | 161117 | 440 | 6.9 |
| CAMA-1 | 161121 | 377 | 7.308 |
| CAL-120 | 161121 | 405 | 0.556 |
| CALU-3 | 161121 | 420 | 7.6 |
| MDA-MB-436 | 161128 | 1050 | 10.35 |
| MDA-MB-436 | 161201 | 1250 | 5.922 |
| HEK293 | 161205 | 2086 | 10.122 |
| PC-3 | 161205 | 560 | 8.01 |
| SW-48 | 161205 | 1220 | 10.78 |
| H358 | 161208 | 790 | 5.664 |

Primers and Probes:

TABLE 4
Primers and probes related to the RAD51 gene

| | NCBI | | SEQ | |
|---|---|---|---|---|
| Gene | Accession | Primer/probe | ID | |
| name | No. | name | NO. | Sequence |
| RAD51 | NM_001164269 | RDS1-F1 | 1 | AAATCTACGACTCTCCCTGTCTTCCT |
| | | RDS1-R1 | 2 | GGCATCTCCCACTCCATCTG |
| | | RDS1-P1 | 3 | TGAAGCTATGTTCGCCATT |
| | | RDS1-F2 | 4 | TGGAGCTAATGGCAATGCAGA |
| | | RDS1-R2 | 5 | CCACACTGCTCTAACCGTGA |
| | | RDS1-P2 | 6 | TTGAAGCAAATGCAGATAC |
| | | RDS1-F3 | 7 | GACTCGCTGATGAGTTTGGTGTA |
| | | RDS1-R3 | 8 | CGCTGCTCCATCCACTTGA |
| | | RDS1-P3 | 9 | CAGTGGTAATCACTAATC |
| | | RDS1-F4 | 10 | GATGCAGCTTGAAGCAAATGC |
| | | RDS1-R4 | 11 | GCATTTATGCCACACTGCTCTAA |
| | | RDS1-P4 | 12 | AAGAAAGCTTTGGCCCACA |
| | | RDS1-F5 | 13 | TGCTGATCCCAAAAAACCTATTG |
| | | RDS1-R5 | 14 | ATTCTGGTTTCCCCTCTTCCTT |
| | | RDS1-P5 | 15 | CATCAACAACCAGATTGTAT |

TABLE 5
Primers and probes related to the XRCC5 gene

| | NCBI | | SEQ | |
|---|---|---|---|---|
| Gene | Accession | Primer/probe | ID | |
| name | No. | name | NO. | Sequence |
| XRCC5 | NM_021141.3 | RDS2-F1 | 16 | CTCCCACCGAGGCACAGT |
| | | RDS2-R1 | 17 | TCAAGGGTGTCTGTCTTCTCATCT |
| | | RDS2-P1 | 18 | CTCCATGAGCTTGGC |
| | | RDS2-F2 | 19 | AGATGAGAAGACAGACACCCTTGA |
| | | RDS2-R2 | 20 | TGCAGCAGACACTGAAATAATCTCT |
| | | RDS2-P2 | 21 | CAACCACCAAAATCCCAA |
| | | RDS2-F3 | 22 | GGACGTGGGCTTTACCATGA |
| | | RDS2-R3 | 23 | AGCAAACACCTGTCGCTGTA |
| | | RDS2-P3 | 24 | CATTCCTGGTATAGAATC |
| | | RDS2-F4 | 25 | ATTCAAGGGTTCCGCTATGGA |
| | | RDS2-R4 | 26 | CAGAGAAGCACTTCCCCTCC |
| | | RDS2-P4 | 27 | CCTTTCTCTAAAGTGGATGAG |
| | | RDS2-F5 | 28 | TGTACAGCGACAGGTGTTTGCT |
| | | RDS2-R5 | 29 | TCAGTGCCATCTGTACCAAACAG |
| | | RDS2-P5 | 30 | AACAAGGATGAGATTGCTTTA |

TABLE 6
Primers and probes related to the RIF1 gene

| | NCBI | | SEQ | |
|---|---|---|---|---|
| Gene | Accession | Primer/probe | ID | |
| name | No. | name | NO. | Sequence |
| RIF1 | NM_018151.4 | RDS3-F1 | 31 | GATCTTCCTAGGGTGGCCGA |
| | | RDS3-R1 | 32 | GCAGACTGACGCTTACCTGA |
| | | RDS3-P1 | 33 | CTGTTGGAGACTTTG |
| | | RDS3-F2 | 34 | CTTGTGCTGAGCTTAGAGCCATT |
| | | RDS3-R2 | 35 | AACAAAGCTATCATGAACAGCAGTG |
| | | RDS3-P2 | 36 | AACATCCGTTAATCAGC |
| | | RDS3-F3 | 37 | TGGATATTCTTAATGGAACTCCAGCT |
| | | RDS3-R3 | 38 | TTCCAAACTGAGAAAGAACCTTTCA |
| | | RDS3-P3 | 39 | TTCTTGGAATGTGGTGTATCA |
| | | RDS3-F4 | 40 | GATTCTAAGATGATGATTACGGAGGAG |
| | | RDS3-R4 | 41 | CAGTCTTCCGTGACATCTTGAGG |
| | | RDS3-P4 | 42 | AATGGACAGTGACATTGT |

TABLE 7
Primers and probes related to the PARPBP gene

| | NCBI | | SEQ | |
|---|---|---|---|---|
| Gene | Accession | Primer/probe | ID | |
| name | No. | name | NO. | Sequence |
| PARPBP | NM_017915.4 | RDS4-F1 | 43 | GGCGTGCTCTTTGTAACTCTGA |
| | | RDS4-R1 | 44 | CCACTGTGCTGTTTGTTGTTCTC |
| | | RDS4-P1 | 45 | ACTCCATGCTCTTGGCA |
| | | RDS4-F2 | 46 | TTTTTGGTGGCCACGTCTTT |
| | | RDS4-R2 | 47 | TGATGGTGGTGGTGCATATCC |
| | | RDS4-P2 | 48 | CAATAGAGCTTGGAGGGAA |
| | | RDS4-F3 | 49 | GGATCCTTCCGCACACTGAA |
| | | RDS4-R3 | 50 | ACAGCCATTATGTGATTAGGGGT |
| | | RDS4-P3 | 51 | GAGTACGTCTTCGGGTCT |
| | | RDS4-F4 | 52 | CAATTCTCAAGAAGGTGTTGTAGCTC |
| | | RDS4-R4 | 53 | GAGATTTTGGCCGAGCAGG |
| | | RDS4-P4 | 54 | TAGCACCACTGACATCA |

TABLE 8
Primers and probes related to the CALM2 gene

| | NCBI | | SEQ | |
|---|---|---|---|---|
| Gene | Accession | Primer/probe | ID | |
| name | No. | name | NO. | Sequence |
| CALM2 | NM_001743.5 | RDS7-F1 | 55 | TGAGATCTCTTGGGCAGAATCC |
| | | RDS7-R1 | 56 | CCATTACCATCAGCATCTACTTCATTA |
| | | RDS7-P1 | 57 | AGAAGCAGAGTTACAGGAC |
| | | RDS7-F2 | 58 | CGGTAGCGCTTGCAGCAT |
| | | RDS7-R2 | 59 | AAAAGCTTCTTTGAATTCTGCAATC |
| | | RDS7-P2 | 60 | CTGACCAACTGACTGAAG |
| | | RDS7-F3 | 61 | GAGCGAGCTGAGTGGTTGTG |
| | | RDS7-R3 | 62 | AGTCAGTTGGTCAGCCATGCT |
| | | RDS7-P3 | 63 | CGTCTCGGAAACCG |

TABLE 9
Primers and probes related to the B2M gene

| | NCBI | Primer/ | SEQ | |
|---|---|---|---|---|
| Gene | Accession | probe | ID | |
| name | No. | name | NO. | Sequence |
| B2M | NM_004048 | RDS8-F1 | 64 | CGCTCCGTGGCCTTAGC |
| | | RDS8-R1 | 65 | AATCTTTGGAGTACGCTG |
| | | | | GATAGC |
| | | RDS8-P1 | 66 | CGCTACTCTCTCTTTC |
| | | RDS8-F2 | 67 | GTATGCCTGCCGTGTGAACC |
| | | RDS8-R2 | 68 | GGCATCTTCAAACCTCCAT |
| | | | | GAT |
| | | RDS8-P2 | 69 | AGTGGGATCGAGACATGTA |
| | | RDS8-F3 | 70 | CTGCCGTGTGAACCATGTG |
| | | RDS8-R3 | 71 | GCTTACATGTCTCGATCCC |
| | | | | ACTT |
| | | RDS8-P3 | 72 | CTTTGTCACAGCCCAAG |

TABLE 10
Primers and probes related to the TBP gene

| | NCBI | | SEQ | |
|---|---|---|---|---|
| Gene | Accession | Primer/probe | ID | |
| name | No. | name | NO. | Sequence |
| TBP | NM_003194 | RDS9-F1 | 73 | CAGGAGCCAAGAGTGAAGAACAGT |
| | | RDS9-R1 | 74 | TGGAAAACCCAACTTCTGTACAACT |
| | | RDS9-P1 | 75 | TGGCAGCAAGAAAA |
| | | RDS9-F2 | 76 | TAACAGGTGCTAAAGTCAGAGCAGAA |
| | | RDS9-R2 | 77 | TCGTCTTCCTGAATCCCTTTAGA |
| | | RDS9-P2 | 78 | AGCATTTGAAAACATCTAC |
| | | RDS9-F3 | 79 | CGAATATAATCCCAAGCGGTTT |
| | | RDS9-R3 | 80 | CCGTGGTTCGTGGCTCTCT |
| | | RDS9-P3 | 81 | CTGCGGTAATCATG |
| | | RDS9-F4 | 82 | CACAGTGAATCTTGGTTGTAAACTTGA |
| | | RDS9-R4 | 83 | AAACCGCTTGGGATTATATTCG |
| | | RDS9-P4 | 84 | TTGCACTTCGTGCCCG |

TABLE 11
Primers and probes related to the GUSB gene

| | NCBI | | SEQ | |
|---|---|---|---|---|
| Gene | Accession | Primer/probe | ID | |
| name | No. | name | NO. | Sequence |
| GUSB | NM_00181 | RDS12-F1 | 85 | GAGTATGGAGCAGAAACGATTGC |
| | | RDS12-R1 | 86 | CAGACTTTTCTGGTACTCTTCAGTGAA |
| | | RDS12-P1 | 87 | TTCACCAGGATCCACCTC |
| | | RDS12-F2 | 88 | GTATGGAGCAGAAACGATTGCA |
| | | RDS12-R2 | 89 | CAGACTTTTCTGGTACTCTTCAGTGAA |
| | | RDS12-P2 | 90 | TTTCACCAGGATCCACC |
| | | RDS12-F3 | 91 | TGGTTGGAGAGCTCATTTGGA |
| | | RDS12-R3 | 92 | ACTCTCGTCGGTGACTGTTCAG |
| | | RDS12-P3 | 93 | TTTTGCCGATTTCATG |

One-step RT-qPCR kit: TaqMan Fast Virus 1-Step Master Mix (Thermo Fisher, Cat. No. 4444432).

Plasmid: Synthesized by GenScript.

2. Methods

I. RNA Extraction Methods

The RNA samples were obtained from cell lines that were simultaneously tested for drug sensitivity, and total RNA was extracted from the cells using the Axygen kit.

II. RT-PCR Method

2.1 8 sets of specific primers and probes were designed and were synthesized by GenScript;

2.2 The plasmids of 8 genes were constructed, and corresponding mRNAs were obtained as standards by in vitro transcription (IVT);

2.3 The IVT RNAs were diluted by a ten-fold gradient to perform a standard curve test on each gene, then the amplification efficiencies were calculated;

2.4 The target gene and the internal reference gene were labeled by two different types of fluorescent probes, FAM and VIC, respectively, to form a dual RT-qPCR reaction system;

2.5 Different thresholds were set in the results of the RT-qPCR reaction, and the corresponding CT values were obtained to calculate ΔCT;

ΔCT=CT₍ₛₐₘₚₗₑ₎−CT_{(reference)} 2.6

2.7 The data analysis of the experimental results includes the CT values of 8 genes and ΔCT in 4 sets of dual RT-qPCR, which is helpful to analyze the different expression levels of each gene in samples and evaluate the DNA repair ability of the sample source.

III. Results of RT-qPCR

The test samples corresponded to the cell lines that had been tested for drug sensitivity. Each gene was tested using the same reagents and instruments in different samples, and the thresholds were consistent. The results are as follows:

TABLE 12
CT values obtained from quantitative analysis of 8 genes

| | Ct Valus | | | | | | | |
|---|---|---|---|---|---|---|---|---|
| Cell lines | RAD51 | XRCC5 | RIF1 | PARPBP | CALM2 | B2M | TBP | GUSB |
| Capan-1 | 26.58 | 23.39 | 23.93 | 25.20 | 19.39 | 18.20 | 23.63 | 23.61 |
| HCC1937 | 26.39 | 24.95 | 23.92 | 26.88 | 20.78 | 19.40 | 25.46 | 23.73 |
| A549 | 26.49 | 23.07 | 23.53 | 24.61 | 19.26 | 20.62 | 23.16 | 22.58 |
| H1299 | 25.83 | 21.94 | 22.29 | 24.62 | 17.84 | 19.55 | 23.18 | 22.49 |
| HT29 | 26.61 | 23.06 | 24.16 | 25.23 | 20.07 | 20.89 | 22.56 | 23.56 |
| H1975 | 29.32 | 26.74 | 26.04 | 24.87 | 24.22 | 24.91 | 28.89 | 26.75 |
| H1650 | 27.99 | 24.95 | 24.05 | 24.91 | 21.27 | 19.98 | 25.95 | 24.55 |
| H2228 | 27.91 | 23.72 | 24.07 | 25.71 | 20.70 | 17.58 | 24.00 | 26.12 |
| MCF-7 | 28.33 | 24.63 | 24.61 | 25.28 | 20.38 | 21.29 | 24.52 | 23.88 |
| HCC95 | 25.52 | 23.30 | 24.69 | 24.91 | 21.86 | 21.44 | 25.88 | 26.83 |
| H520 | 27.37 | 24.50 | 25.06 | 24.29 | 22.02 | 21.31 | 24.92 | 25.45 |
| HCC827 | 30.63 | 24.95 | 24.28 | 26.16 | 23.00 | 22.62 | 28.06 | 24.42 |
| HCC4006 | 28.97 | 23.88 | 24.94 | 26.80 | 20.60 | 19.19 | 24.12 | 24.40 |
| H3122 | 24.66 | 21.77 | 22.13 | 24.29 | 17.92 | 19.65 | 21.55 | 21.94 |
| KYSE450 | 26.28 | 22.37 | 23.92 | 23.35 | 22.29 | 20.76 | 24.26 | 23.49 |
| CAMA-1 | 29.21 | 25.51 | 24.22 | 26.72 | 20.87 | 21.48 | 25.67 | 24.81 |
| HEK-293 | 27.35 | 25.05 | 24.96 | 26.00 | 21.42 | 22.28 | 23.88 | 23.87 |
| T84 | 27.34 | 25.18 | 24.59 | 24.65 | 20.94 | 19.34 | 23.99 | 22.37 |
| PC-3 | 26.30 | 25.46 | 23.73 | 25.59 | 22.42 | 23.33 | 25.13 | 24.35 |
| MDA-MB-436 | 26.56 | 24.70 | 23.90 | 26.04 | 20.59 | 18.65 | 24.57 | 22.51 |
| Cal-120 | 28.06 | 24.71 | 25.00 | 26.09 | 20.19 | 21.09 | 25.73 | 24.90 |
| H2444 | 26.06 | 25.18 | 25.28 | 27.50 | 22.11 | 21.21 | 26.39 | 24.89 |

IV. RDS Value Calculation

For any cell line, the average of the CT values of the four internal reference genes were first calculated, and then the CT values of the RAD51, XRCC5, RIF1, and PARPBP genes were subtracted by the average value respectively to obtain the ΔCT values of the four genes. The same method was used to obtain the ΔCT values of 4 genes in all cell lines.

For all 22 samples, statistical analysis was performed on all ΔCT values of each gene, and the averages and variances of the ΔCT values of the genes were obtained.

For any cell line, the Z value of each gene=(ΔCT value of the gene−the average value of the ΔCT value of the gene)/the variance of the ΔCT value of the gene, and the RDS value of the cell line is the sum of the negative Z value of all 4 genes. In this way, the following results were obtained.

TABLE 13
The Z values and RDS values of the cell lines

| | Z values | | | | |
|---|---|---|---|---|---|
| Cell lines | RAD51 | XRCC5 | RIF1 | PARPBP | RDS values |
| Capan-1 | 0.57 | 0.61 | 1.24 | 0.80 | −3.22 |
| HCC1937 | −0.44 | 1.05 | −0.03 | 1.17 | −1.75 |
| A549 | 0.35 | 0.07 | 0.58 | 0.27 | −1.27 |
| H1299 | 0.34 | −0.43 | −0.09 | 0.70 | −0.52 |
| HT29 | 0.16 | −0.31 | 0.87 | 0.44 | −1.16 |
| H1975 | −1.14 | −1.07 | −1.96 | −2.77 | 6.95 |
| H1650 | 0.32 | 0.43 | −0.55 | −0.56 | 0.36 |
| H2228 | 0.90 | 0.03 | 0.41 | 0.54 | −1.89 |
| MCF-7 | 0.90 | 0.53 | 0.55 | −0.03 | −1.95 |
| HCC95 | −2.37 | −2.37 | −1.03 | −1.28 | 7.04 |
| H520 | −0.52 | −0.54 | 0.03 | −1.31 | 2.34 |
| HCC827 | 1.12 | −1.20 | −2.06 | −0.79 | 2.93 |
| HCC4006 | 1.73 | 0.22 | 1.40 | 1.29 | −4.63 |
| H3122 | −0.17 | −0.09 | 0.30 | 0.82 | −0.85 |
| KYSE450 | −0.80 | −1.98 | −0.43 | −1.45 | 4.65 |
| CAMA-1 | 1.05 | 0.73 | −0.66 | 0.47 | −1.58 |
| HEK-293 | −0.11 | 0.61 | 0.55 | 0.23 | −1.28 |
| T84 | 0.80 | 1.98 | 1.47 | 0.13 | −4.37 |
| PC-3 | −1.62 | 0.07 | −1.87 | −0.69 | 4.11 |
| MDA-MB-436 | 0.27 | 1.57 | 0.80 | 1.11 | −3.75 |
| Ca1-120 | 0.35 | 0.14 | 0.47 | 0.20 | −1.17 |
| H2444 | −1.68 | −0.06 | 0.02 | 0.70 | 1.02 |

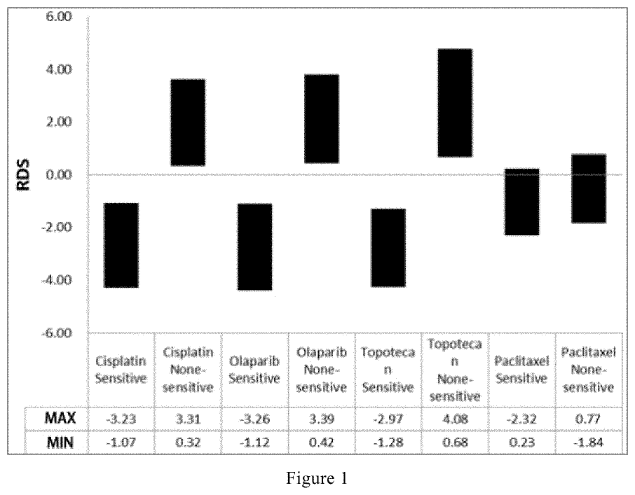

The RDS values of each drug-sensitive and non-sensitive sample were analyzed. The results are shown in Table 14 and FIG. 1.

TABLE 14
Comparison of RDS values of sensitive and non-sensitive cell lines

| Drugs | | | | |
|---|---|---|---|---|
| Cell lines | Cisplatin | Olaparib | Topotecan | Paclitaxel |
| Sensitive | −2.15 ± 1.08* | −2.19 ± 1.07 | −2.13 ± 0.85 | −1.04 ± 1.27 |
| samples | | | | |
| Non-sensitive | 1.81 ± 1.49 | 1.9 ± 1.48 | 2.38 ± 1.7 | −0.54 ± 1.31 |
| samples | | | | |

*Datas are mean ± SE.

The results show that cell lines sensitive to DNA damage drugs have lower RDS values, and no significant difference in the RDS values of tumor cell lines, whether sensitive or not, to paclitaxel, a non-DNA damage drug, further confirms that RDS can guide cancer treatment.

### Example 3

1. Experimental Design

The inventors collected 300 formalin-fixed paraffin-embedded tumor tissue specimens from invasive breast cancer patients.

Inclusion Criteria:

All patients have triple negative (ER/PR IHC test was 0 and HER2 IHC 0-1 or FISH<2.0) breast cancer. 150 patients received platinum neoadjuvant chemotherapy, of which 50 patients achieved complete pathological remission (pCR or Miller-Payne grade 5); 150 patients received ACT neoadjuvant chemotherapy, of which 50 patients achieved pathological complete remission.

All study subjects: Wax blocks originated from patients with invasive breast cancer who underwent total mastectomy without radiation therapy and had complete pathological diagnostic data, including HE and IHC4 staining results and 5 years of complete follow-up data.

The wax block should not have been sliced too many times, and at least six 10 μm thick slices can be produced.

Wax samples have IHC test results and FISH results (when HER2 IHC 2).

Exclusion Criteria:

Wax samples have been stored for too long (>10 years)

After the wax sample was sliced, the tumor tissue content was too small (20%).

According to the inclusion and exclusion criteria, the patient information, pathological diagnosis, and survival data were collected. Finally, 128 cases were included.

Human breast cancer wax samples were collected and sliced. Each sample required six 10 μm rolls.

RDS Determining of Breast Cancer

The RNA samples were extracted from the slices, and then were purified to be tested for RNA concentration and purity. If the quality of a RNA sample meets the standard then proceed to the next step, otherwise the sample is rejected. RNA samples can be stored at 2-8° C. if tested on the same day, otherwise RNA samples need to be stored at −80° C.

Primer design tool was used to design primers according to the genes' sequences, and RNA samples were detected by quantitative PCR. The Z value of each candidate gene was calculated.

Based on the analysis of the test results, the breast cancer RDS value was obtained.

Statistical analysis was performed to assess the predictive RDS value of breast cancer on pCR in TNBC patients.

pCR Predicting:

RDS value of breast cancer predicts pCR: RDS value of breast cancer is used as a continuous variable, and a linear regression analysis is performed to evaluate its correlation with pCR.

TABLE 15
Basic data

| clinical | Samples | Samples without | Samples with |
|---|---|---|---|
| information | (N = 128) | transfer (N = 119) | transfer (N = 9) |
| TNM | N (%) | N | N |
| 1 | 7 (5.5) | 7 | 0 |
| 2 | 21 (16.4) | 21 | 0 |
| 3 | 15 (11.7) | 13 | 2 |
| 4 | 2 (1.6) | 0 | 2 |
| Unclear | 2 (1.6) | 2 | 0 |
| Missing data | 81 (63.2) | 76 | 5 |
| Grade | | | |
| 1 | 0 (0) | 0 | 0 |
| 2 | 15 (11.7) | 14 | 1 |
| 3 | 24 (18.7) | 21 | 3 |
| Cannot be graded | 45 (35.2) | 42 | 3 |
| Missing data | 44 (34.4) | 42 | 2 |
| Pathological type | | | |
| ductal carcinoma in | 2 (1.6) | 2 | 0 |
| situ (DCIS) | | | |
| Infitrating ductal | 63 (49.2) | 56 | 7 |
| carcinoma (IDC) | | | |
| Others | 7 (5.5) | 7 | 0 |
| Missing data | 56 (43.7) | 54 | 2 |
| P-M | [symbol] | | |
| | grade | | |
| 1 | 5 (3.9) | 5 | 0 |
| 2 | 19 (14.8) | 15 | 4 |
| 3 | 28 (21.9) | 26 | 2 |
| 4 | 19 (14.8) | 18 | 1 |
| 5 | 57 (44.6) | 55 | 2 |
| pCR-1 | | | |
| pCR | 57 (44.5) | 55 | 2 |
| Non-pCR | 71 (55.5) | 64 | 7 |
| Whether lymph node | | | |
| with cancer cells | | | |
| YES | 36 (28.1) | 31 | 5 |
| NO | 87 (68.0) | 83 | 4 |
| Missing data | 5 (3.9) | 5 | 0 |
| pCR-2 | | | |
| V | 48 (37.5) | 46 | 2 |
| Non-pCR | 79 (61.7) | 72 | 7 |
| Missing data | 1 (0.8) | 1 | 0 |
| Age | 48.3 ± 11.6 | 48.2 ± 11.6 | |

pCR-1: P-M grade 5; pCR-2: P-M grade 5 + lymph node without tumor cells.

2. Differences in Gene Expression Levels Under Different P-M Grades

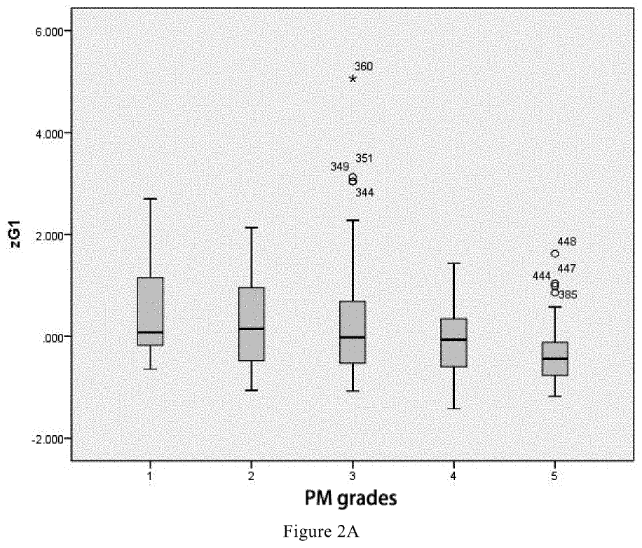

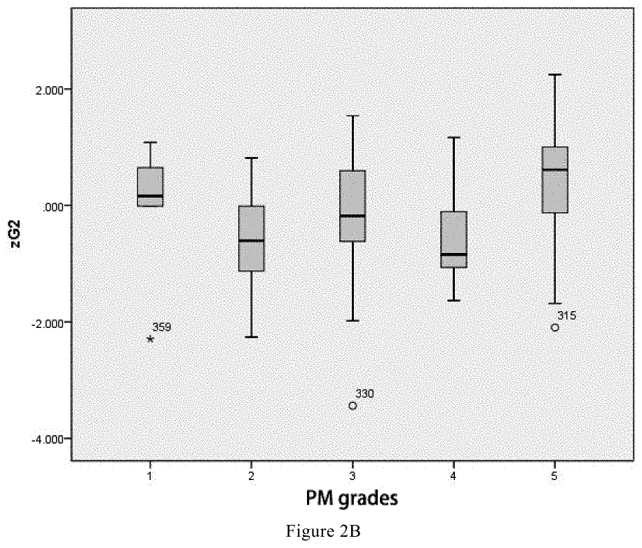

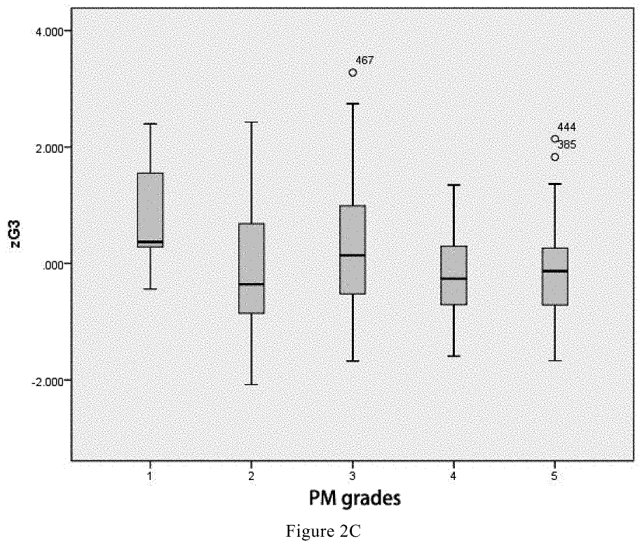

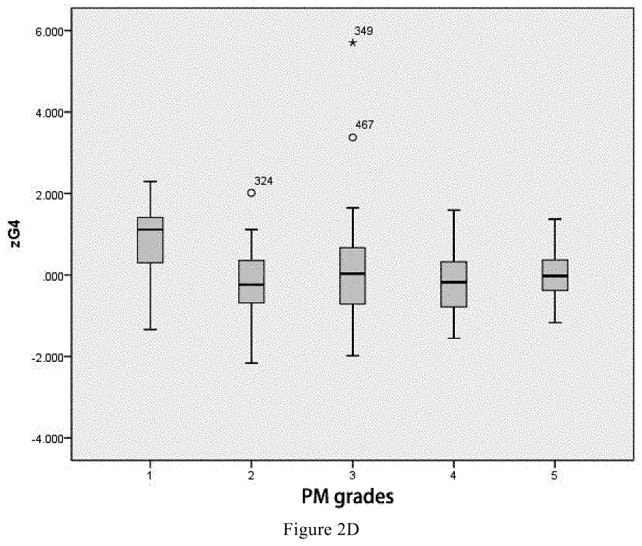

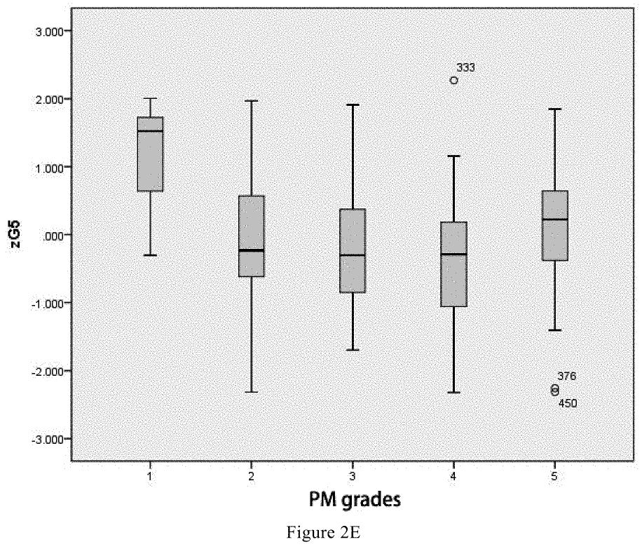

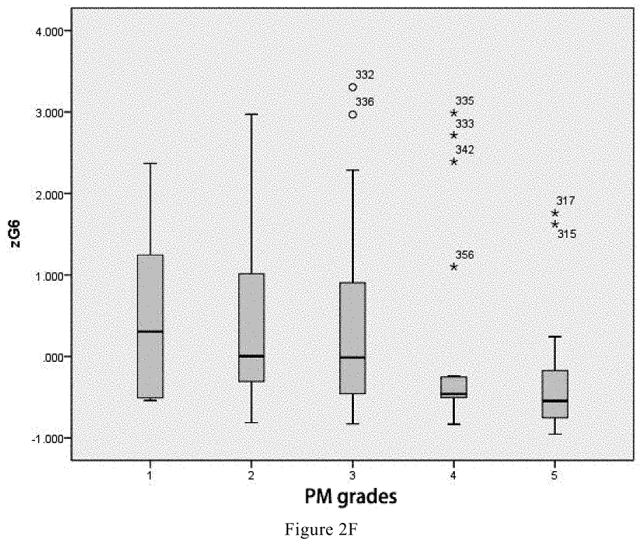

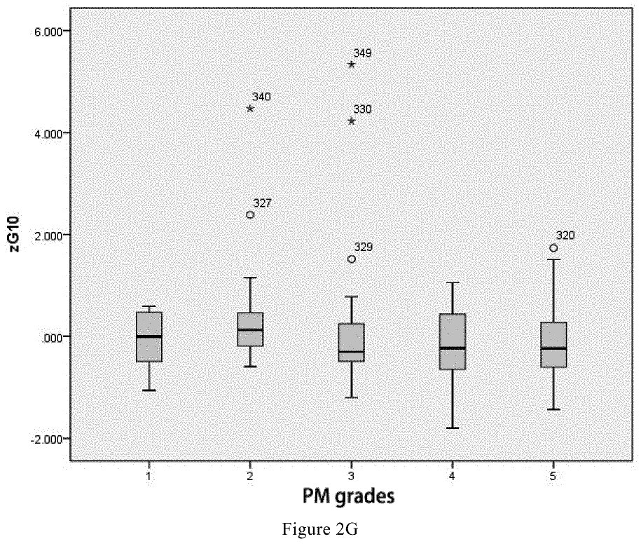

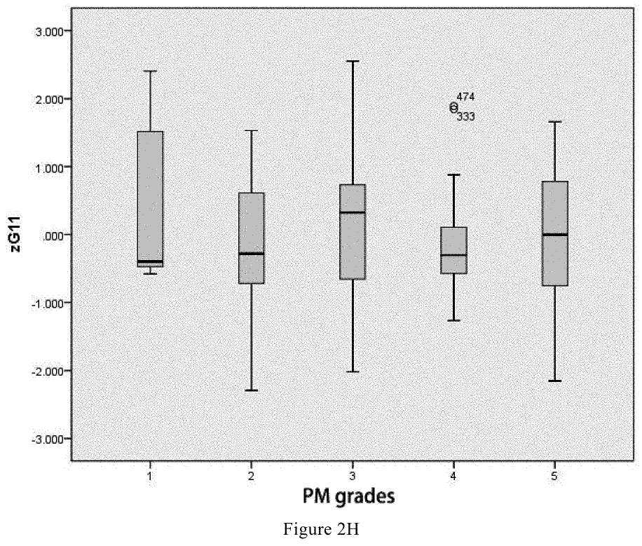

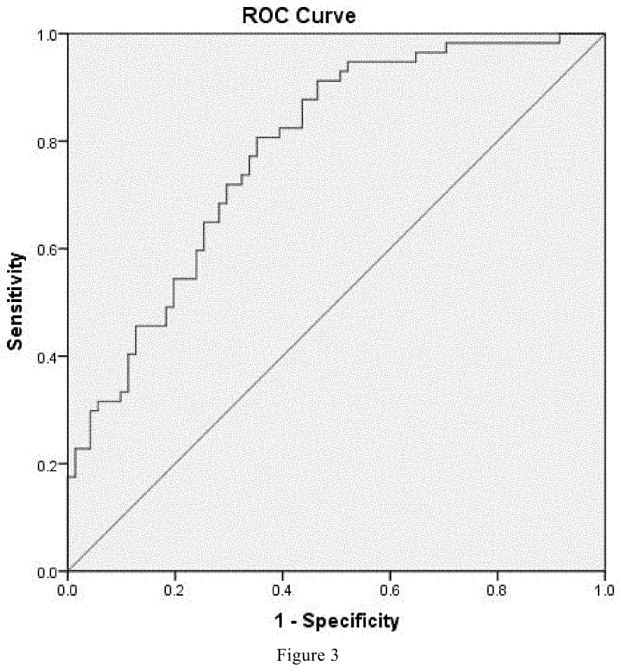

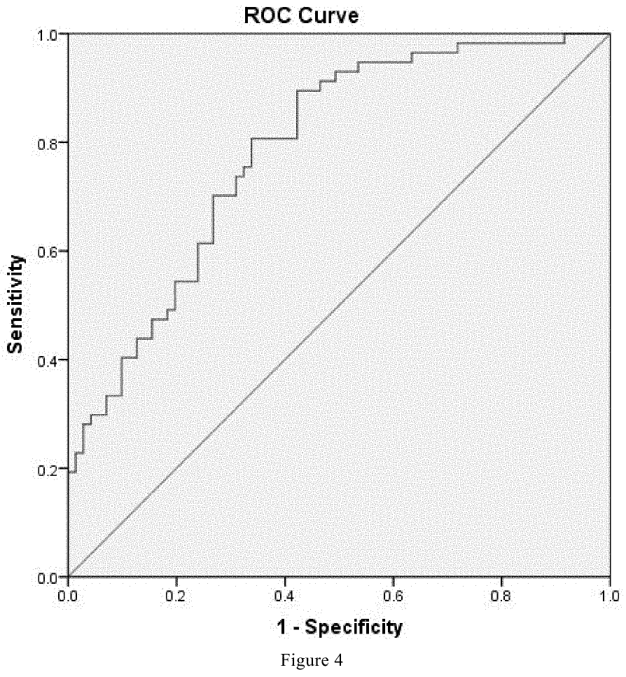

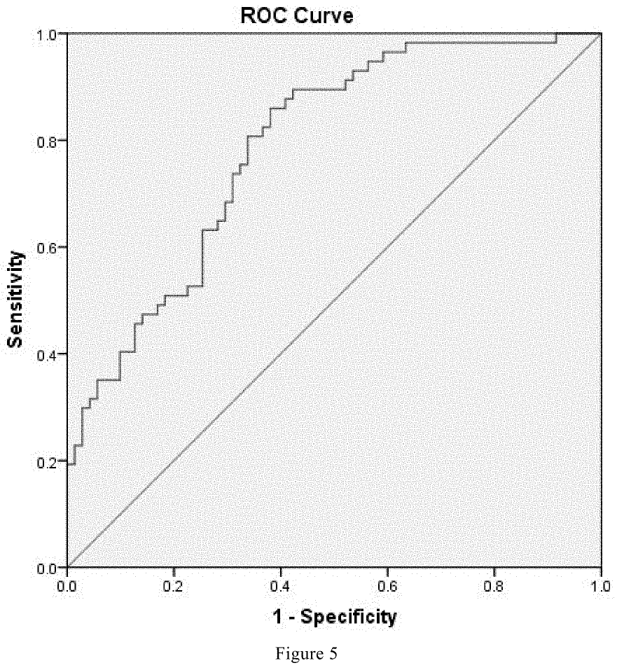

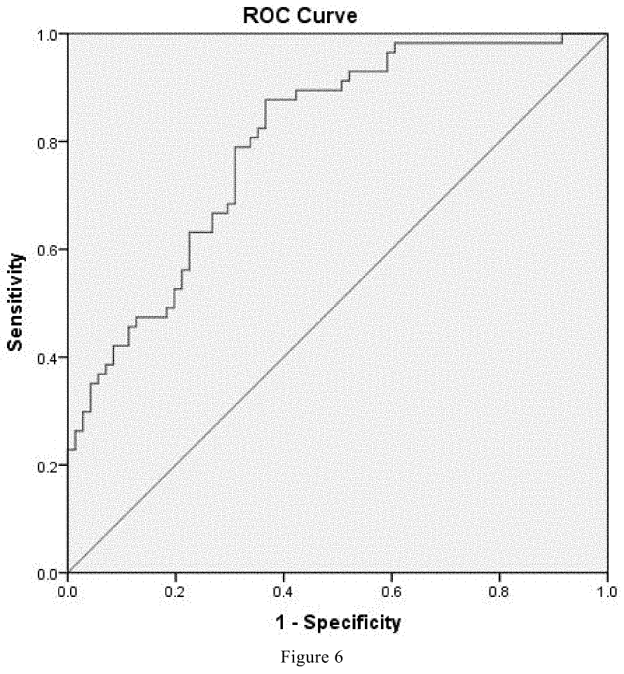

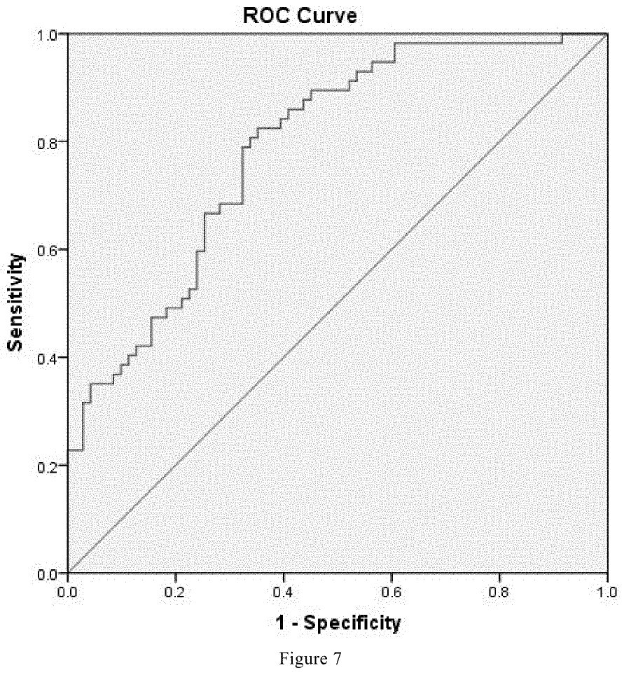

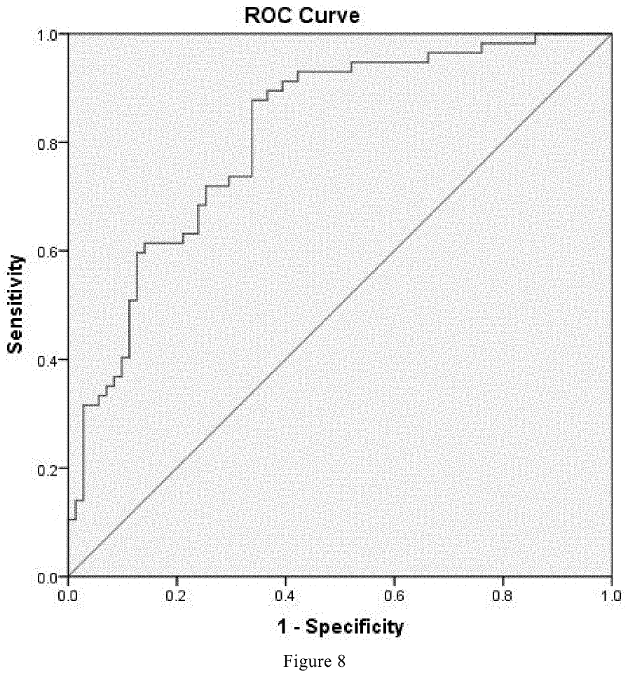

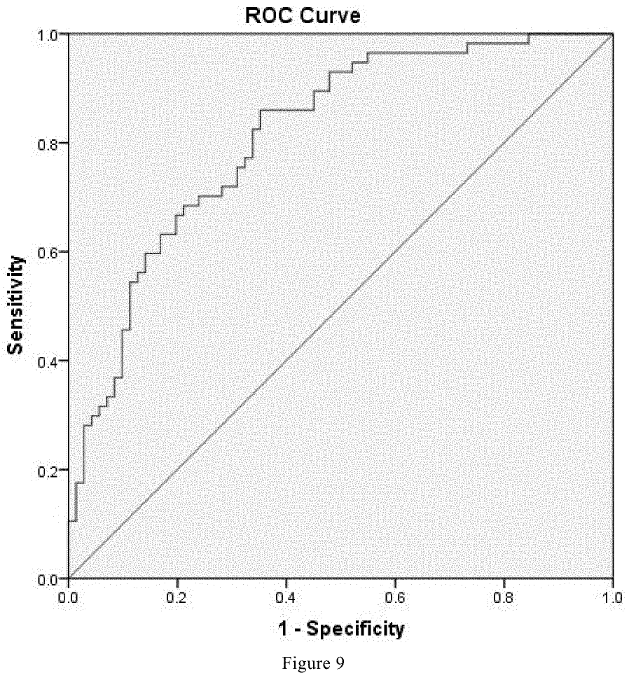

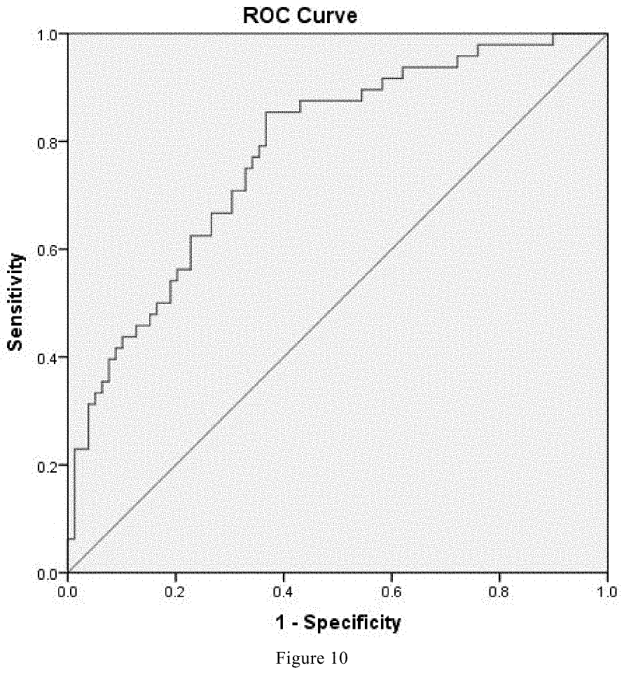

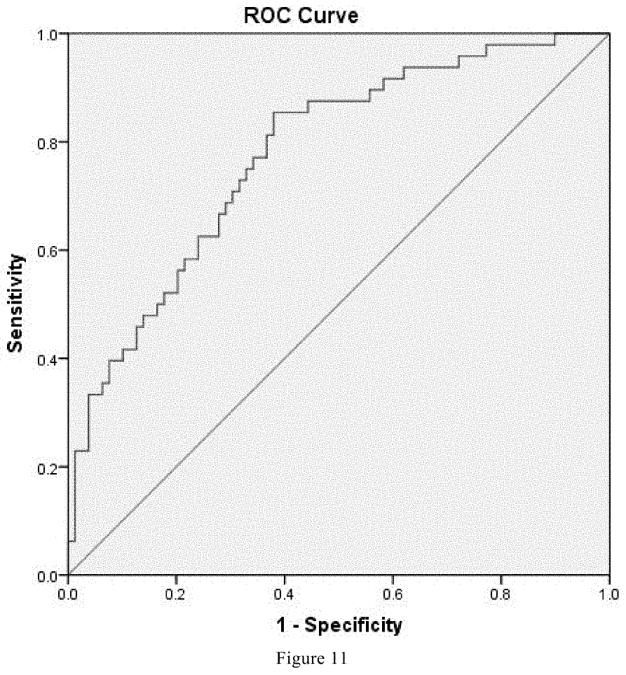

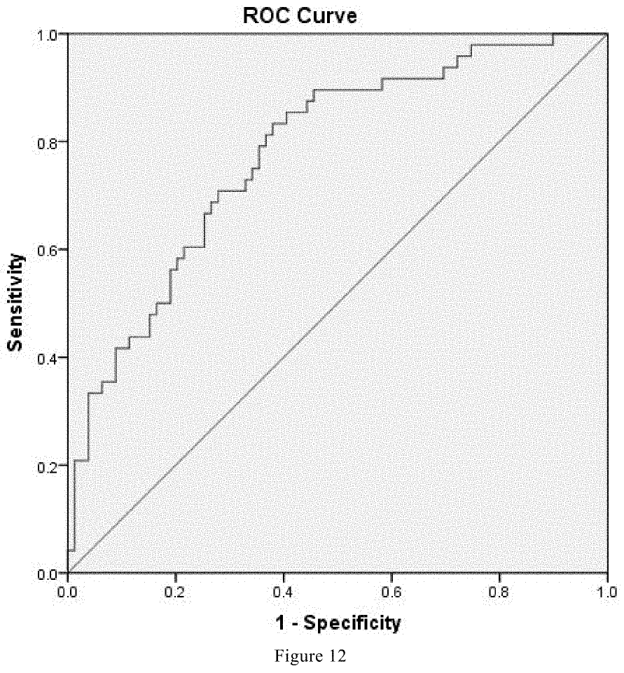

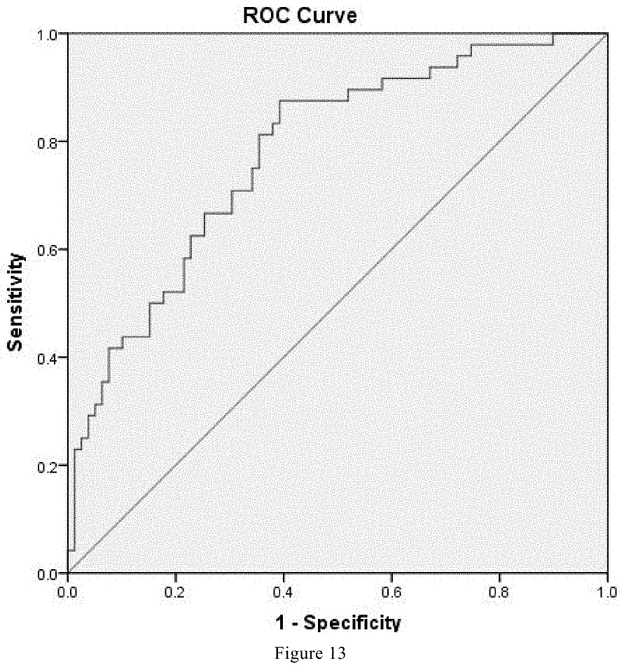

FIG. 2 shows the boxplots of the expression levels of the genes z-G1 (RAD51), z-G2 (XRCC5), z-G3 (RIF1), z-G4 (PARPBP), z-G5 (PARP1), z-G6 (BRCA1), z-G10 (c-Met) and z-G11 (E2F1) under different P-M grades. Univariate analysis of variance showed that the z-G1, z-G2, z-G3, z-G5, z-G6 gene levels were statistically different under different P-M grades (Table 16).

TABLE 16
Differences in gene levels under different P-M grades

| genes | P-M1(n = 5) | P-M2(n = 19) | P-M3(n = 28) | P-M4(n = 19) | P-M5(n = 57) | P |
|---|---|---|---|---|---|---|
| z-G1 | 0.625 ± 1.335 | 0.237 ± 0.896 | 0.476 ± 1.511 | −0.083 ± 0.752 | −0.333 ± 0.577^{abc} | 0.003 |
| z-G2 | −0.084 ± 1.308 | −0.581 ± 0.850 | −0.118 ± 1.108 | −0.549 ± 0.808 | 0.455 ± 0.827^{bcd} | <0.001 |
| z-G3 | 0.836 ± 1.128 | −0.029 ± 1.170 | 0.368 ± 1.184 | −0.208 ± 0.758^{a} | −0.163 ± 0.844^{ac} | 0.045 |
| z-G4 | 0.756 ± 1.370 | −0.222 ± 0.952 | 0.218 ± 1.519 | −0.202 ± 0.879 | −0.014 ± 0.611 | 0.206 |
| z-G5 | 1.118 ± 0.945 | −0.091 ± 1.123^{a} | −0.160 ± 0.908^{a} | −0.325 ± 1.121^{a} | 0.128 ± 0.906^{a} | 0.037 |
| z-G6 | 0.574 ± 1.241 | 0.526 ± 1.163 | 0.345 ± 1.132 | 0.084 ± 1.236 | −0.410 ± 0.516^{abcd} | <0.001 |
| z-G10 | −0.098 ± 0.686 | 0.480 ± 1.189 | 0.153 ± 1.440 | −0.211 ± 0.873^{b} | −0.153 ± 0.650^{b} | 0.124 |
| z-G11 | 0.494 ± 1.377 | −0.200 ± 1.094 | 0.151 ± 1.091 | −0.119 ± 0.876 | 0.007 ± 0.934 | 0.584 |

^{a}shows comparison with PM1, p < 0.05;
^{b}shows comparison with PM2, p < 0.05;
^{c}shows comparison with PM3, p < 0.05;
^{d}shows comparison with PM4, p < 0.05.

2.3 Correlation Between P-M and Gene Expression Levels

Spearman correlation analysis shows that there was statistically significant correlation between z-G1, z-G2, z-G6 gene levels and P-M grades, among which z-G2, z-G4, z-G5 gene levels were positively correlated with P-M.

TABLE 17
Correlation between P-M grades and gene
expression levels

| | Correlation | |
|---|---|---|
| genes | coefficient | P |
| z-G1 | −0.299 | 0.001 |
| z-G2 | 0.357 | <0.001 |
| z-G6 | −0.408 | <0.001 |
| z-G10 | −0.160 | 0.071 |
| z-G3 | −0.137 | 0.124 |
| z-G5 | 0.067 | 0.454 |
| z-G4 | 0.006 | 0.948 |
| z-G11 | −0.001 | 0.991 |

3. Establishment of Random Forest Models to Determine Gene Weights

The variables initially included in the model are: z-G1 (RAD51), z-G2 (XRCC5), z-G3 (RIF1), z-G4 (PARPBP), z-G5 (PARP1), z-G6 (BRCA1), Z-G10 (c-Met), z-G11 (E2F1), age. Taking into account other pathological features such as TNM staging, Grade classification, pathological type, etc., are serious missed and are limited by the sample size so that haven't been included in the model. At the same time, this experiment only targets triple-negative breast cancer, with fixed ER, PR, and HER2 status. Therefore, only age and the expression level of each gene were included.

3.1 Preliminary Results of the Establishment of the pCR-1 Prediction Model

The accuracy, sensitivity and specificity are the highest when the ratio of the training set to the test set is 6:4. If the variables with a normalized importance score <20 are removed, the variables that are finally included in the random forest model are only 3 genes: z.G1, z.G2, z.G6. In order to increase the accuracy of prediction, the variables with importance score <10 can be removed, and the variables included in the random forest model are z.G1, z.G2, z.G4, z.G5, z.G6.

TABLE 18

| | Rank of | | Evaluation indicators | | | | |
|---|---|---|---|---|---|---|---|
| training | the | standardized | | | | Positive | Negative |
| set: | variable | importance | | | | predictive | predictive |
| test set | importance | score | accuracy | sensitivity | specificity | value | value |
| 5:5 | z.G2 | 100.000 | 0.698 | 0.714 | 0.686 | 0.645 | 0.750 |
| | z.G6 | 79.863 | | | | | |
| | z.G1 | 39.840 | | | | | |
| | z.G4 | 31.662 | | | | | |
| | z.G5 | 18.953 | | | | | |
| | z.G11 | 12.756 | | | | | |
| | age | 10.447 | | | | | |
| | z.G10 | 5.662 | | | | | |
| | z.G3 | 0.000 | | | | | |
| 6:4 | z.G2 | 100.000 | 0.78 | 0.773 | 0.786 | 0.739 | 0.815 |
| | z.G6 | 49.430 | | | | | |
| | z.G1 | 31.496 | | | | | |
| | z.G5 | 12.994 | | | | | |
| | z.G4 | 12.581 | | | | | |
| | z.G3 | 7.855 | | | | | |
| | z.G11 | 7.599 | | | | | |
| | age | 5.293 | | | | | |
| | z.G10 | 0.000 | | | | | |
| 7:3 | z.G2 | 100.00 | 0.737 | 0.706 | 0.762 | 0.706 | 0.762 |
| | z.G6 | 91.36 | | | | | |
| | z.G1 | 70.36 | | | | | |
| | z.G4 | 42.44 | | | | | |
| | z.G10 | 20.62 | | | | | |
| | z.G11 | 15.94 | | | | | |
| | z.G3 | 12.76 | | | | | |
| | z.G5 | 12.30 | | | | | |
| | age | 0.00 | | | | | |
| 8:2 | z.G2 | 100.000 | 0.68 | 0.636 | 0.714 | 0.636 | 0.714 |
| | z.G6 | 82.015 | | | | | |
| | z.G1 | 56.691 | | | | | |
| | z.G5 | 16.885 | | | | | |
| | z.G3 | 9.042 | | | | | |
| | z.G10 | 5.479 | | | | | |
| | z.G4 | 4.075 | | | | | |
| | z.G11 | 1.975 | | | | | |
| | age | 0.000 | | | | | |
| 9:1 | z.G2 | 100.000 | 0.667 | 0.600 | 0.714 | 0.600 | 0.714 |
| | z.G6 | 67.672 | | | | | |
| | z.G1 | 54.158 | | | | | |
| | z.G5 | 29.341 | | | | | |
| | z.G3 | 18.183 | | | | | |
| | z.G4 | 17.089 | | | | | |
| | z.G10 | 7.821 | | | | | |
| | z.G11 | 5.660 | | | | | |
| | age | 0.000 | | | | | |

3.2 Final Results of Model Building

3.2.1 Results of the Models Incorporating 5 Genes

The accuracies, sensitivities, and specificities of the model are higher under various ratios of the training set to the test set, so the gene weight for each model can be calculated. The calculation principle of the weights is: obtaining the importance score of each variable according to the model (unstandardized, or, if standardized, the weight cannot be calculated because the score of the least important variable is 0), calculating the final weights according to proportions of the importance scores thus the sum of the final weights is 1. Considering that the genes of G2, G5, and G4 are positively correlated with P-M grades, the weights need to be changed to negative numbers by multiplying −1.

Example: When the ratio of the training set to the test set is 5:5, the weight of z.G2 is:

[1÷(−10.442756+7.917480+5.150769−4.928693−3.170395)]*(−10.442756)=1.9078423

TABLE 19

| | Rank of | | | Evaluation indicators | | | | |
|---|---|---|---|---|---|---|---|---|
| training | the | | | | | | Positive | Negative |
| set: | variable | importance | | | | | predictive | predictive |
| test set | importance | score | Weights | accuracy | sensitivity | specificity | value | value |
| 5:5 | z.G2 | −10.442756 | 1.9078423 | 0.762 | 0.750 | 0.771 | 0.724 | 0.794 |
| | z.G6 | 7.917480 | −1.4464863 | | | | | |
| | z.G1 | 5.150769 | −0.9410212 | | | | | |
| | z.G4 | −4.928693 | 0.9004490 | | | | | |
| | z.G5 | −3.170395 | 0.5792162 | | | | | |
| 6:4 | z.G2 | −13.545782 | 1.8206667 | 0.72 | 0.773 | 0.679 | 0.654 | 0.792 |
| | z.G6 | 8.968040 | −1.2053798 | | | | | |
| | z.G1 | 6.370646 | −0.8562682 | | | | | |
| | z.G4 | −4.995132 | 0.6713876 | | | | | |
| | z.G5 | −4.237784 | 0.5695937 | | | | | |
| 7:3 | z.G2 | −11.298340 | 2.9992700 | 0.790 | 0.706 | 0.857 | 0.800 | 0.783 |
| | z.G6 | 11.029066 | −2.9277882 | | | | | |
| | z.G1 | 9.091019 | −2.4133121 | | | | | |
| | z.G4 | −7.110227 | 1.8874888 | | | | | |
| | z.G5 | −5.478548 | 1.4543415 | | | | | |
| 8:2 | z.G2 | −13.510081 | 3.4459578 | 0.72 | 0.727 | 0.714 | 0.667 | 0.769 |
| | z.G6 | 12.282383 | −3.1328142 | | | | | |
| | z.G1 | 10.962153 | −2.7960688 | | | | | |
| | z.G5 | −7.038045 | 1.7951636 | | | | | |
| | z.G4 | −6.616969 | 1.6877616 | | | | | |
| 9:1 | z.G2 | −15.429872 | 2.0106046 | 0.833 | 0.800 | 0.857 | 0.800 | 0.857 |
| | z.G6 | 12.374767 | −1.6125061 | | | | | |
| | z.G1 | 12.260766 | −1.5976511 | | | | | |
| | z.G4 | −8.612708 | 1.1222873 | | | | | |
| | z.G5 | −8.267198 | 1.0772653 | | | | | |

3.2.2 Results of the Models Incorporating 3 Genes

The sensitivities of the model are higher when ratios of the training set to the test sets are 5:5 and 7:3, so the gene weights under this model can be calculated. 5 The calculation principle of the weights is consistent with the above. In general, the accuracy and sensitivity of models that incorporate only three genes are lower than those that incorporate five genes.

TABLE 20

| | | | | Evaluation indicators | | | | |
|---|---|---|---|---|---|---|---|---|
| training | Rank of the | | | | | | Positive | Negative |
| set: | variable | importance | | | | | predictive | predictive |
| test set | importance | score | Weights | accuracy | sensitivity | specificity | value | value |
| 5:5 | z.G2 | −12.847173 | −2.1358314 | 0.698 | 0.679 | 0.714 | 0.655 | 0.735 |
| | z.G6 | 11.236503 | 1.8680589 | | | | | |
| | z.G1 | 7.625739 | 1.2677725 | | | | | |
| 6:4 | z.G2 | −17.816281 | −7.1663458 | 0.64 | 0.682 | 0.607 | 0.577 | 0.708 |
| | z.G6 | 12.154383 | 4.8889278 | | | | | |
| | z.G1 | 8.148002 | 3.2774180 | | | | | |
| 7:3 | z.G2 | −16.04913 | −1.3606527 | 0.658 | 0.706 | 0.619 | 0.600 | 0.722 |
| | z.G6 | 15.03225 | 1.2744411 | | | | | |
| | z.G1 | 12.81205 | 1.0862116 | | | | | |
| 8:2 | z.G2 | −18.85587 | −1.4891782 | 0.68 | 0.636 | 0.714 | 0.636 | 0.714 |
| | z.G1 | 15.76929 | 1.2454097 | | | | | |
| | z.G6 | 15.74851 | 1.2437685 | | | | | |
| 9:1 | z.G2 | −22.05840 | −1.7279537 | 0.583 | 0.600 | 0.571 | 0.500 | 0.667 |
| | z.G6 | 17.88773 | 1.4012426 | | | | | |
| | z.G1 | 16.93629 | 1.3267111 | | | | | |

4. Establishment of RDS System

If the gene's weight is still positively correlated with P-M, then the established RDS value needs to be multiplied by −1 to ensure that RDS value is negatively correlated with P-M, otherwise it doesn't need to be multiplied by −1.

4.1 Models that Incorporated 5 Genes

Training set: Test set=5:5

RDS₁=−1*(1.9078423*z.G2-1.4464863*z.G6−0.9410212*z.G1+0.9004490*z.G4+0.5792162*z.G5)

Training set: Test set=6:4

RDS₂=−1*(1.8206667*z.G2−1.2053798*z.G6−0.8562682*z.G1+0.6713876*z.G4+0.5695937*z.G5)

Training set: Test set=7:3

RDS₃=−1*(2.9992700*z.G2−2.9277882*z.G6−2.4133121*z.G1+1.8874888*z.G4+1.4543415*z.G5)

Training set: Test set=8:2

RDS₄=−1*(3.4459578*z.G2−3.1328142*z.G6−2.7960688*z.G1+1.6877616*z.G4+1.7951636*z.G5)

Training set: Test set=9:1

RDS₅=−1*(2.0106046*z.G2−1.6125061*z.G6−1.5976511*z.G1+1.1222873*z.G4+1.0772653*z.G5)

4.2 Models that Incorporated 3 Genes

Training set: Test set=5:5

RDS₆=1.8680589*z.G6−2.1358314*z.G2+1.2677725*z.G1

Training set: Test set=7:3

RDS₇=1.2744411*z.G6−1.3606527*z.G2+1.0862116*z.G1

5. RDS Predicts pCR-Logistic Regression

5.1 Logistic Regression Predicts the Risk of pCR

Logistic regression found that the RDS value was significantly correlated with the probability of occurrence of pCR-1, and that the decrease in RDS value increased the probability of occurrence of pCR. The RDS₄ model has the highest accuracy and the RDS₆ has the highest AUC.

TABLE 21
Risk analysis of RDS₁ and pCR-1

| | Odds ratio | | | Hosmer-Lemeshow |
|---|---|---|---|---|
| Variables | (95% CI) | P | Accuracy | test [×2 (P)] |
| RDS₁ | 1.503 (1.262-1.789) | <0.001 | 70.3 | 5.183 (0.738) |
| age | 1.009 (0.973-1.045) | 0.635 | | |

TABLE 22
Risk analysis of RDS₂ and pCR-1

| | Odds ratio | | | Hosmer-Lemeshow |
|---|---|---|---|---|
| Variables | (95% CI) | P | Accuracy | test [×2 (P)] |
| RDS₂ | 1.586 (1.306-1.926) | <0.001 | 71.1 | 8.171 (0.417) |
| age | 1.008 (0.973-1.045) | 0.644 | | |

TABLE 23
Risk analysis of RDS₃ and pCR-1

| | Odds ratio | | | Hosmer-Lemeshow |
|---|---|---|---|---|
| Variables | (95% CI) | P | Accuracy | test [×2 (P)] |
| RDS₃ | 1.586 (1.306-1.926) | <0.001 | 71.1 | 11.002 (0.202) |
| age | 1.008 (0.973-1.045) | 0.644 | | |

TABLE 24
Risk analysis of RDS₄ and pCR-1

| | Odds ratio | | | Hosmer-Lemeshow |
|---|---|---|---|---|
| Variables | (95% CI) | P | Accuracy | test [×2 (P)] |
| RDS₄ | 1.244 (1.136-1.362) | <0.001 | 71.9 | 8.152 (0.419) |
| age | 1.010 (0.974-1.047) | 0.594 | | |

TABLE 25
Risk analysis of RDS₅ and pCR-1

| | Odds ratio | | | Hosmer-Lemeshow |
|---|---|---|---|---|
| Variables | (95% CI) | P | Accuracy | test [×2 (P)] |
| RDS₅ | 1.433 (1.232-1.668) | <0.001 | 69.5 | 6.623 (0.578) |
| age | 1.010 (0.974-1.047) | 0.594 | | |

TABLE 26
Risk analysis of RDS₆ and pCR-1

| | Odds ratio | | | Hosmer-Lemeshow |
|---|---|---|---|---|
| Variables | (95% CI) | P | Accuracy | test [×2 (P)] |
| RDS₆ | 1.565 (1.300-1.883) | <0.001 | 72.7 | 12.278 (0.139) |
| age | 1.004 (0.968-1.041) | 0.849 | | |

TABLE 27
Risk analysis of RDS₇ and pCR-1

| | Odds ratio | | | Hosmer-Lemeshow |
|---|---|---|---|---|
| Variables | (95% CI) | P | Accuracy | test [×2 (P)] |
| RDS₇ | 1.917 (1.465-2.509) | <0.001 | 73.4 | 6.034 (0.643) |
| age | 1.003 (0.967-1.040) | 0.870 | | |

TABLE 28
Distribution of 7 systems

| Variables | Mean | SE | P₅₀ (P₂₅, P₇₅) |
|---|---|---|---|
| RDS₁ | −0.008615738 | −0.702159416 | −0.702 (−2.488, 2060) |
| RDS₂ | −0.007996746 | −0.605954413 | −0.606 (−2.300, 1.845) |
| RDS₃ | −0.012426881 | −1.257691228 | −1.258 (−4.577, 3.727) |
| RDS₄ | −0.012284822 | −1.624126638 | −1.624 (−5.049, 4.157) |
| RDS₅ | −0.009779668 | −0.865191444 | −0.865 (−3.038, 2.385) |
| RPS₆ | 0.002971957 | −0.660292689 | −0.660 (−2.721, 1.678) |
| RPS₇ | 0.003286237 | −0.541729238 | −0.542 (−1.897, 0.955) |

6. One-Way Analysis of Variance for Differences in RDS Under Different PM Grades

TABLE 29

| Variables | PM1(n = 5) | PM2(n = 19) | PM3(n = 28) | PM4(n = 19) | PM5(n = 57) | P |
|---|---|---|---|---|---|---|
| RDS₁ | 0.251 ± 3.871 | 2.344 ± 3.440 | 1.069 ± 4.107 | 1.460 ± 3.059 | −1.835 ± 2.444^{bcd} | <0.001 |
| RDS₂ | 0.236 ± 3.398 | 2.095 ± 3.082 | 0.983 ± 3.651 | 1.350 ± 2.679 | −1.670 ± 2.225^{bcd} | <0.001 |
| RDS₃ | 0.389 ± 6.908 | 4.405 ± 6.692 | 2.335 ± 7.939 | 2.545 ± 5.804 | −3.526 ± 4.538^{bcd} | <0.001 |
| RDS₄ | 0.553 ± 7.362 | 4.850 ± 7.439 | 2.738 ± 8.683 | 2.846 ± 6.252 | −3.986 ± 5.040^{bcd} | <0.001 |
| RDS₅ | 0.041 ± 4.098 | 2.742 ± 4.197 | 1.482 ± 5.004 | 1.683 ± 3.533 | −2.228 ± 2.939^{bcd} | <0.001 |
| RDS₆ | 2.045 ± 4.763 | 2.523 ± 3.918 | 1.500 ± 4.438 | 1.224 ± 3.612 | −2.159 ± 2.421^{abcd} | <0.001 |
| RDS₇ | 1.526 ± 3.293 | 1.718 ± 2.743 | 1.117 ± 3.163 | 0.764 ± 2.500 | −1.503 ± 1.640^{abcd} | <0.001 |

^{a}shows comparison with PM1, p < 0.05;
^{b}shows comparison with PM2, p < 0.05;
^{c}shows comparison with PM3, p < 0.05;
^{d}shows comparison with PM4, p < 0.05.

7. Establishment of pCR-2 Prediction Model

7.1 Establishing a Random Forest Model to Determine Gene Weights

The variables initially included in the random forest model are still z-G1, z-G2, z-G3, z-G4, z-G5, z-G6, z-G10, z-G11 and age.

7.2 Preliminary Results of Establishment of the Model

7.2.1 When the ratios of training set to the test set are 7:3 and 8:2, the model has higher accuracies, sensitivities, and specificities. The variables with importance scores <15 are removed and the variables included in the random forest model are: z.G1, z.G2, z.G5, z.G6, z.G10.

TABLE 30

| | Rank of | | Evaluation indicators | | | | |
|---|---|---|---|---|---|---|---|
| training | the | | | | | Positive | Negative |
| set:test | variable | importance | | | | predictive | predictive |
| set | importance | score | accuracy | sensitivity | specificity | value | value |
| 5:5 | age | 100.000 | 0.698 | 0.333 | 0.923 | 0.727 | 0.692 |
| | z.G2 | 76.013 | | | | | |
| | z.G6 | 62.794 | | | | | |
| | z.G1 | 60.531 | | | | | |
| | z.G5 | 29.819 | | | | | |
| | z.G3 | 8.754 | | | | | |
| | z.G11 | 5.208 | | | | | |
| | z.G4 | 4.758 | | | | | |
| | z.G10 | 0.000 | | | | | |
| 6:4 | z.G2 | 100.000 | 0.7 | 0.421 | 0.871 | 0.667 | 0.711 |
| | age | 87.711 | | | | | |
| | z.G1 | 56.056 | | | | | |
| | z.G5 | 50.675 | | | | | |
| | z.G6 | 43.262 | | | | | |
| | z.G10 | 13.320 | | | | | |
| | z.G4 | 11.605 | | | | | |
| | z.G3 | 0.607 | | | | | |
| | z.G11 | 0.000 | | | | | |
| 7:3 | z.G2 | 100.00 | 0.703 | 0.571 | 0.783 | 0.615 | 0.750 |
| | z.G6 | 70.250 | | | | | |
| | age | 39.119 | | | | | |
| | z.G1 | 23.915 | | | | | |
| | z.G10 | 19.905 | | | | | |
| | z.G5 | 16.770 | | | | | |
| | z.G4 | 3.907 | | | | | |
| | z.G3 | 1.976 | | | | | |
| | z.G11 | 0.000 | | | | | |
| 8:2 | z.G2 | 100.000 | 0 708 | 0.556 | 0.800 | 0.625 | 0.750 |
| | z.G6 | 73.973 | | | | | |
| | z.G5 | 23.021 | | | | | |
| | age | 18.770 | | | | | |
| | z.G10 | 17.227 | | | | | |
| | z.G1 | 15.722 | | | | | |
| | z.G11 | 11.219 | | | | | |
| | z.G4 | 7.743 | | | | | |
| | z.G3 | 0.000 | | | | | |
| 9:1 | z.G2 | 100.000 | 0.636 | 0.500 | 0.714 | 0.500 | 0.714 |
| | z.G6 | 63.804 | | | | | |
| | z.G1 | 43.538 | | | | | |
| | z.G5 | 31.321 | | | | | |
| | z.G10 | 19.210 | | | | | |
| | z.G3 | 16.227 | | | | | |
| | z.G4 | 16.033 | | | | | |
| | age | 8.322 | | | | | |
| | z.G11 | 0.000 | | | | | |

7.2.2 Treat age as a categorical variable, divide it into <48 and ≥48, and establish a random forest model. The difference in the standardized importance scores of each variable is small, and it is not easy to screen.

TABLE 31

| | Rank of | | Evaluation indicators | | | | |
|---|---|---|---|---|---|---|---|
| training | the | | | | | Positive | Negative |
| set:test | variable | importance | | | | predictive | predictive |
| set | importance | score | accuracy | sensitivity | specificity | value | value |
| 5:5 | z.G2 | 100.00 | 0.746 | 0.542 | 0.872 | 0.722 | 0.756 |
| | z.G6 | 87.02 | | | | | |
| | z.G1 | 72.79 | | | | | |
| | z.G5 | 58.93 | | | | | |
| | z.G11 | 43.72 | | | | | |
| | z.G4 | 42.36 | | | | | |
| | z.G10 | 39.29 | | | | | |
| | z.G3 | 29.85 | | | | | |
| | age | 0.00 | | | | | |
| 6:4 | z.G2 | 100.00 | 0.72 | 0.474 | 0.871 | 0.692 | 0.730 |
| | z.G1 | 85.72 | | | | | |
| | z.G5 | 84.12 | | | | | |
| | z.G6 | 72.31 | | | | | |
| | z.G4 | 61.79 | | | | | |
| | z.G10 | 59.34 | | | | | |
| | z.G3 | 52.19 | | | | | |
| | z.G11 | 47.49 | | | | | |
| | age | 0.00 | | | | | |
| 7:3 | z.G2 | 100.00 | 0.703 | 0.571 | 0.783 | 0.615 | 0.750 |
| | z.G6 | 87.38 | | | | | |
| | z.G5 | 60.54 | | | | | |
| | z.G1 | 58.57 | | | | | |
| | z.G10 | 52.63 | | | | | |
| | z.G3 | 50.06 | | | | | |
| | z.G4 | 47.63 | | | | | |
| | z.G11 | 43.64 | | | | | |
| | age | 0.00 | | | | | |
| 8:2 | z.G2 | 100.00 | 0.75 | 0.667 | 0.800 | 0.667 | 0.800 |
| | z.G6 | 84.39 | | | | | |
| | z.G5 | 47.86 | | | | | |
| | z.G1 | 45.52 | | | | | |
| | z.G10 | 39.83 | | | | | |
| | z.G11 | 36.37 | | | | | |
| | z.G4 | 31.30 | | | | | |
| | z.G3 | 30.84 | | | | | |
| | age | 0.00 | | | | | |
| 9:1 | z.G2 | 100.00 | 0.727 | 0.750 | 0.714 | 0.600 | 0.833 |
| | z.G6 | 86.12 | | | | | |
| | z.G1 | 73.15 | | | | | |
| | z.G5 | 68.59 | | | | | |
| | z.G10 | 59.63 | | | | | |
| | z.G3 | 58.28 | | | | | |
| | z.G4 | 56.85 | | | | | |
| | z.G11 | 46.63 | | | | | |
| | age | 0.00 | | | | | |

7.3 the Results of the Established Model

The accuracies, sensitivities, and specificities of the model are higher when the ratios of the training set to the test set are 8:2 and 9:1, so the gene weight for each model can be calculated. The calculation principle of the weights is: obtaining the importance score of each variable according to the model (unstandardized, or, if standardized, the weight cannot be calculated because the score of the least important variable is 0), calculating the final weights according to proportions of the importance scores thus the sum of the final weights is 1. Considering that the genes of G2 and G5 are positively correlated with P-M grades, the weights need to be changed to negative numbers.

TABLE 32

| | Rank of | | | Evaluation indicators | | | | |
|---|---|---|---|---|---|---|---|---|
| training | the | | | | | | Positive | Negative |
| set: | variable | importance | | | | | predictive | predictive |
| test set | importance | score | Weights | accuracy | sensitivity | specificity | value | value |
| 5:5 | z.G2 | −7.229296 | −1.7869991 | 0.730 | 0.500 | 0.872 | 0.706 | 0.739 |
| | z.G1 | 6.273305 | 1.5506891 | | | | | |
| | z.G6 | 6.043832 | 1.4939660 | | | | | |
| | z.G5 | −5.439050 | −1.3444708 | | | | | |
| | z.G10 | 4.396704 | 1.0868148 | | | | | |
| 6:4 | z.G2 | −8.868242 | −2.1714242 | 0.68 | 0.526 | 0.774 | 0.588 | 0.727 |
| | z.G1 | 7.624512 | 1.8668920 | | | | | |
| | z.G5 | −6.886296 | −1.6861369 | | | | | |
| | z.G6 | 6.774232 | 1.6586976 | | | | | |
| | z.G10 | 5.439861 | 1.3319715 | | | | | |
| 7:3 | z.G2 | −11.045661 | −1.8846270 | 0.649 | 0.500 | 0.739 | 0.539 | 0.708 |
| | z.G6 | 9.198058 | 1.5693862 | | | | | |
| | z.G1 | 7.637908 | 1.3031911 | | | | | |
| | z.G10 | 7.006253 | 1.1954172 | | | | | |
| | z.G5 | −6.935631 | −1.1833675 | | | | | |
| 8:2 | z.G2 | −12.482474 | −2.1128981 | 0.708 | 0.667 | 0.733 | 0.600 | 0.786 |
| | z.G6 | 11.775126 | 1.9931659 | | | | | |
| | z.G5 | −8.545787 | −1.4465384 | | | | | |
| | z.G1 | 7.973882 | 1.3497325 | | | | | |
| | z.G10 | 7.187003 | 1.2165381 | | | | | |
| 9:1 | z.G2 | −13.690096 | −1.6731665 | 0.727 | 0.750 | 0.714 | 0.600 | 0.833 |
| | z.G6 | 11.715879 | 1.4318830 | | | | | |
| | z.G1 | 10.645709 | 1.3010898 | | | | | |
| | z.G5 | −9.318313 | −1.1388590 | | | | | |
| | z.G10 | 8.828969 | 1.0790527 | | | | | |

7.4. Establishment of RDS System

If the gene's weight is still positively correlated with P-M, then the established RDS value needs to be multiplied by −1 to ensure that RDS value is negatively correlated with P-M, otherwise it doesn't need to be multiplied by −1.

7.4.1 Models that Incorporated 5 Genes

Training set: Test set=5:5

RDS₈=1.4939660*z.G6−1.7869991*z.G2−1.3444708*z.G5+1.5506891*z.G1+1.0868148*z.G10

Training set: Test set=6:4

RDS₉=1.6586976*z.G6−2.1714242*z.G2−1.6861369*z.G5+1.8668920*z.G1+1.3319715*z.G10

Training set: Test set=8:2

RDS₁₀=1.9931659*z.G6−2.1128981*z.G2−1.4465384*z.G5+1.3497325*z.G1+1.2165381*z.G10

Training set: Test set=9:1

RDS₁₁=1.4318830*z.G6−1.6731665*z.G2+1.3010898*z.G1−1.1388590*z.G5+1.0790527*z.G10

7.5. RDS Predicts pCRr—Logistic Regression

7.5.1 Logistic Regression Predicts the Risk of pCR-2

Logistic regression found that all the RDS were significantly correlated with the probability of occurrence of pCR-2, and that the decrease in RDS value increased the probability of occurrence of pCR.

The RDS₉ and RDS₁₁ model has higher accuracies and the RDS₈ has the highest AUC.

TABLE 33
Risk analysis of RDS₈ and pCR-2

| | Odds ratio | | | Hosmer-Lemeshow |
|---|---|---|---|---|
| Variables | (95% CI) | P | Accuracy | test [×2 (P)] |
| RDS₈ | 1.434 (1.215-1.691) | <0.001 | 70.9 | 11.166 (0.192) |
| age | 1.018 (0.982-1.056) | 0.320 | | |

TABLE 34
Risk analysis of RDS₉ and pCR-2

| | Odds ratio | | | Hosmer-Lemeshow |
|---|---|---|---|---|
| Variables | (95% CI) | P | Accuracy | test [×2 (P)] |
| RDS₉ | 1.345 (1.174-1.542) | <0.001 | 71.7 | 9.764 (0.282) |
| age | 1.018 (0.982-1.055) | 0.322 | | |

TABLE 35
Risk analysis of RDS₁₀ and pCR-2

| | Odds ratio | | | Hosmer-Lemeshow |
|---|---|---|---|---|
| Variables | (95% CI) | P | Accuracy | test [×2 (P)] |
| RDS₁₀ | 1.367 (1.182-1.580) | <0.001 | 70.1 | 10.787 (0.214) |
| age | 1.019 (0.983-1.056) | 0.303 | | |

TABLE 36
Risk analysis of RDS₁₁ and pCR-2

| | Odds ratio | | | Hosmer-Lemeshow |
|---|---|---|---|---|
| Variables | (95% CI) | P | Accuracy | test [×2 (P)] |
| RDS₁₁ | 1.474 (1.232-1.764) | <0.001 | 71.7 | 9.718 (0.285) |
| age | 1.018 (0.982-1.055) | 0.324 | | |

TABLE 37
Distribution of 4 systems

| Variables | Mean | SE | P50 (P25, P75) |
|---|---|---|---|
| RDS₈ | 0.023608545 | 4.308 | −0.652 (−2.679, 1.604) |
| RDS₉ | 0.026666982 | 5.139 | −0.791 (−3.104, 1.743) |
| RDS₁₀ | 0.027362254 | 4.932 | −1.066 (−3.130, 1.912) |
| RDS₁₁ | 0.021913723 | 3.989 | −0.696 (−2.427, 1.438) |

4. One-Way Analysis of Variance for Differences in RDS Under Different PM Grades

TABLE 38

| Variable | PM1(n = 5) | PM2(n = 19) | PM3(n = 28) | PM4(n = 19) | PM5(n = 56) | P |
|---|---|---|---|---|---|---|
| RDS₈ | 0.368 ± 3.836 | 2.836 ± 4.768 | 1.845 ± 5.163 | 1.185 ± 3.306 | −2.266 ± 2.667^{bcd} | <0.001 |
| RDS₉ | 0.286 ± 4.478 | 3.370 ± 5.673 | 2.190 ± 6.172 | 1.443 ± 3.870 | −2.693 ± 3.231^{bcd} | <0.001 |
| RDS₁₀ | 0.429 ± 4.519 | 3.312 ± 5.550 | 1.996 ± 5.786 | 1.428 ± 3.944 | −2.582 ± 3.050^{bcd} | <0.001 |
| RDS₁₁ | 0.397 ± 3.644 | 2.655 ± 4.447 | 1.657 ± 4.784 | 1.073 ± 3.129 | −2.079 ± 2.436^{bc} | <0.001 |

^{a}shows comparison with PM1, p < 0.05;
^{b}shows comparison with PM2, p < 0.05;
^{c}shows comparison with PM3, p < 0.05;
^{d}shows comparison with PM4, p < 0.05.

All documents mentioned in the present application are hereby incorporated by reference in their entireties as if the disclosures. In addition, it is to be understood that various modifications and changes may be made by those skilled in the art in the form of the appended claims.

1. A method for treating human cancer, comprising:
predicting the sensitivity of tumor cells or tissues in a cancer patient to DNA damage therapy; and
administering DNA damage therapy to said cancer patient,
wherein the step of predicting the sensitivity of tumor cells or tissues in cancer patients to DNA damage therapy comprises obtaining a DNA recombination deficiency score (RDS) of said tumor cells or tissues, and said RDS value is calculated based on the expression levels of a plurality of DNA repair related genes,
said plurality of DNA repair related genes comprising at least 4 gene chosen from the group of RAD51, XRCC5, RIF1, PARPBP, PARP1, BRCA1, c-Met and E2F1.

2. The method of claim 1, wherein said DNA damage therapy is selected from chemotherapy or radiotherapy.

3. (canceled)

4. The method of claim 2, wherein said chemotherapy comprises chemical agent selected from the group of platinum compounds, DNA cross-linking agents, topoisomerase inhibitors, and PARP inhibitors.

5. The method of claim 4, wherein said platinum compound is cisplatin or carboplatin.

6. The method of claim 4, wherein said DNA cross-linking agent is cisplatin, said topoisomerase inhibitor is topotecan.

7. The method of claim 4, wherein said PARP inhibitor is olaparib.

8-11. (canceled)

12. The method of claim 1, wherein said RDS value is calculated by the following steps:
(1) subtracting the average value of the expression levels of a first DNA repair related gene in a patient population with the expression level of said gene, and dividing by the standard deviation of the expression level of said gene in the patient population to obtain the Z value of said gene;
(2) repeating step (1) to obtain the Z values of a second DNA repair related gene;
(3) multiplying the Z value of said first DNA repair related gene by its weight, and multiplying the Z value of said second DNA repair related gene by its weight, and
(4) summing the Z values of said first and second DNA repair related genes to arrive at said RDS value.

13. The method of claim 12, wherein said expression levels of the first and second DNA repair related genes are relative to the expression level of one or more reference genes.

14-16. (canceled)

17. The method of claim 1, wherein if said RDS value is lower than a preset cutoff value or falls within a preset interval, a DNA damage therapy is administered to said cancer patient.

18. The method of claim 1, wherein said cancer is breast cancer.

19-24. (canceled)

25. The method of claim 1, wherein said expression levels are obtained by nucleic acid hybridization and/or amplification.

26-30. (canceled)

31. A diagnostic kit, comprising:
primers for amplifying transcripts of a plurality of DNA repair related genes and/or probes capable of hybridizing with transcripts of said DNA repair related genes, or
antibodies selectively immunoreactive with proteins expressed by said plurality of DNA repair related genes,
wherein said plurality of DNA repair related genes comprises at least 4 gene chosen from the group of RAD51, XRCC5, RIF1, PARPBP, PARP1, BRCA1, c-Met and E2F1.

32-41. (canceled)

42. The kit of claim 31, wherein said kit further comprises primers for amplifying the transcripts of one or more reference genes or probes for hybridizing with transcripts of said one or more reference genes, or antibodies selectively immunoreactive with proteins expressed by the one or more reference genes.

43-51. (canceled)

52. The kit of claim 31, wherein said probe is labeled with a label.

53. The kit of claim 52, wherein said probe is labeled with radioisotope, fluorescein, biotin, enzyme, enzyme substrate, ligand, or antibody.

54. The kit of claim 52, wherein said probe is bound to a solid support.

55-62. (canceled)

63. A method for predicting pCR of a breast cancer patient after receiving neoadjuvant therapy, comprising:
determining the expression levels of a plurality of DNA repair related genes, said plurality of DNA repair related genes comprising at least four genes chosen from the group of RAD51, XRCC5, RIF1, PARPBP, PARP1, BRCA1, c-Met and E2F.

64-66. (canceled)

67. The method of claim 13, wherein said one or more reference genes are chosen from the group of CALM2, B2M, TBP and GUSB.

68. The kit of claim 42, wherein said one or more reference genes are chosen from the group of CALM2, B2M, TBP and GUSB.


SEQUENCE LISTING (93 sequences)

SEQ ID NO: 1 - DNA, 26 bp, Artificial Sequence
  RDS1-F1
aaatctacgactctccctgtcttcct

SEQ ID NO: 2 - DNA, 20 bp, Artificial Sequence
  RDS1-R1
ggcatctcccactccatctg

SEQ ID NO: 3 - DNA, 19 bp, Artificial Sequence
  RDS1-P1
tgaagctatgttcgccatt

SEQ ID NO: 4 - DNA, 21 bp, Artificial Sequence
  RDS1-F2
tggagctaatggcaatgcaga

SEQ ID NO: 5 - DNA, 20 bp, Artificial Sequence
  RDS1-R2
ccacactgctctaaccgtga

SEQ ID NO: 6 - DNA, 19 bp, Artificial Sequence
  RDS1-P2
ttgaagcaaatgcagatac

SEQ ID NO: 7 - DNA, 23 bp, Artificial Sequence
  RDS1-F3
gactcgctgatgagtttggtgta

SEQ ID NO: 8 - DNA, 19 bp, Artificial Sequence
  RDS1-R3
cgctgctccatccacttga

SEQ ID NO: 9 - DNA, 18 bp, Artificial Sequence
  RDS1-P3
cagtggtaatcactaatc

SEQ ID NO: 10 - DNA, 21 bp, Artificial Sequence
  RDS1-F4
gatgcagcttgaagcaaatgc

SEQ ID NO: 11 - DNA, 23 bp, Artificial Sequence
  RDS1-R4
gcatttatgccacactgctctaa

SEQ ID NO: 12 - DNA, 19 bp, Artificial Sequence
  RDS1-P4
aagaaagctttggcccaca

SEQ ID NO: 13 - DNA, 23 bp, Artificial Sequence
  RDS1-F5
tgctgatcccaaaaaacctattg

SEQ ID NO: 14 - DNA, 22 bp, Artificial Sequence
  RDS1-R5
attctggtttcccctcttcctt

SEQ ID NO: 15 - DNA, 20 bp, Artificial Sequence
  RDS1-P5
catcaacaaccagattgtat

SEQ ID NO: 16 - DNA, 18 bp, Artificial Sequence
  RDS2-F1
ctcccaccgaggcacagt

SEQ ID NO: 17 - DNA, 24 bp, Artificial Sequence
  RDS2-R1
tcaagggtgtctgtcttctcatct

SEQ ID NO: 18 - DNA, 15 bp, Artificial Sequence
  RDS2-P1
ctccatgagcttggc

SEQ ID NO: 19 - DNA, 24 bp, Artificial Sequence
  RDS2-F2
agatgagaagacagacacccttga

SEQ ID NO: 20 - DNA, 25 bp, Artificial Sequence
  RDS2-R2
tgcagcagacactgaaataatctct

SEQ ID NO: 21 - DNA, 18 bp, Artificial Sequence
  RDS2-P2
caaccaccaaaatcccaa

SEQ ID NO: 22 - DNA, 20 bp, Artificial Sequence
  RDS2-F3
ggacgtgggctttaccatga

SEQ ID NO: 23 - DNA, 20 bp, Artificial Sequence
  RDS2-R3
agcaaacacctgtcgctgta

SEQ ID NO: 24 - DNA, 18 bp, Artificial Sequence
  RDS2-P3
cattcctggtatagaatc

SEQ ID NO: 25 - DNA, 21 bp, Artificial Sequence
  RDS2-F4
attcaagggttccgctatgga

SEQ ID NO: 26 - DNA, 20 bp, Artificial Sequence
  RDS2-R4
cagagaagcacttcccctcc

SEQ ID NO: 27 - DNA, 21 bp, Artificial Sequence
  RDS2-P4
cctttctctaaagtggatgag

SEQ ID NO: 28 - DNA, 22 bp, Artificial Sequence
  RDS2-F5
tgtacagcgacaggtgtttgct

SEQ ID NO: 29 - DNA, 23 bp, Artificial Sequence
  RDS2-R5
tcagtgccatctgtaccaaacag

SEQ ID NO: 30 - DNA, 21 bp, Artificial Sequence
  RDS2-P5
aacaaggatgagattgcttta

SEQ ID NO: 31 - DNA, 20 bp, Artificial Sequence
  RDS3-F1
gatcttcctagggtggccga

SEQ ID NO: 32 - DNA, 20 bp, Artificial Sequence
  RDS3-R1
gcagactgacgcttacctga

SEQ ID NO: 33 - DNA, 15 bp, Artificial Sequence
  RDS3-P1
ctgttggagactttg

SEQ ID NO: 34 - DNA, 23 bp, Artificial Sequence
  RDS3-F2
cttgtgctgagcttagagccatt

SEQ ID NO: 35 - DNA, 25 bp, Artificial Sequence
  RDS3-R2
aacaaagctatcatgaacagcagtg

SEQ ID NO: 36 - DNA, 17 bp, Artificial Sequence
  RDS3-P2
aacatccgttaatcagc

SEQ ID NO: 37 - DNA, 26 bp, Artificial Sequence
  RDS3-F3
tggatattcttaatggaactccagct

SEQ ID NO: 38 - DNA, 25 bp, Artificial Sequence
  RDS3-R3
ttccaaactgagaaagaacctttca

SEQ ID NO: 39 - DNA, 21 bp, Artificial Sequence
  RDS3-P3
ttcttggaatgtggtgtatca

SEQ ID NO: 40 - DNA, 27 bp, Artificial Sequence
  RDS3-F4
gattctaagatgatgattacggaggag

SEQ ID NO: 41 - DNA, 23 bp, Artificial Sequence
  RDS3-R4
cagtcttccgtgacatcttgagg

SEQ ID NO: 42 - DNA, 18 bp, Artificial Sequence
  RDS3-P4
aatggacagtgacattgt

SEQ ID NO: 43 - DNA, 22 bp, Artificial Sequence
  RDS4-F1
ggcgtgctctttgtaactctga

SEQ ID NO: 44 - DNA, 23 bp, Artificial Sequence
  RDS4-R1
ccactgtgctgtttgttgttctc

SEQ ID NO: 45 - DNA, 17 bp, Artificial Sequence
  RDS4-P1
actccatgctcttggca

SEQ ID NO: 46 - DNA, 20 bp, Artificial Sequence
  RDS4-F2
tttttggtggccacgtcttt

SEQ ID NO: 47 - DNA, 21 bp, Artificial Sequence
  RDS4-R2
tgatggtggtggtgcatatcc

SEQ ID NO: 48 - DNA, 19 bp, Artificial Sequence
  RDS4-P2
caatagagcttggagggaa

SEQ ID NO: 49 - DNA, 20 bp, Artificial Sequence
  RDS4-F3
ggatccttccgcacactgaa

SEQ ID NO: 50 - DNA, 23 bp, Artificial Sequence
  RDS4-R3
acagccattatgtgattaggggt

SEQ ID NO: 51 - DNA, 18 bp, Artificial Sequence
  RDS4-P3
gagtacgtcttcgggtct

SEQ ID NO: 52 - DNA, 26 bp, Artificial Sequence
  RDS4-F4
caattctcaagaaggtgttgtagctc

SEQ ID NO: 53 - DNA, 19 bp, Artificial Sequence
  RDS4-R4
gagattttggccgagcagg

SEQ ID NO: 54 - DNA, 17 bp, Artificial Sequence
  RDS4-P4
tagcaccactgacatca

SEQ ID NO: 55 - DNA, 22 bp, Artificial Sequence
  RDS7-F1
tgagatctcttgggcagaatcc

SEQ ID NO: 56 - DNA, 27 bp, Artificial Sequence
  RDS7-R1
ccattaccatcagcatctacttcatta

SEQ ID NO: 57 - DNA, 19 bp, Artificial Sequence
  RDS7-P1
agaagcagagttacaggac

SEQ ID NO: 58 - DNA, 18 bp, Artificial Sequence
  RDS7-F2
cggtagcgcttgcagcat

SEQ ID NO: 59 - DNA, 25 bp, Artificial Sequence
  RDS7-R2
aaaagcttctttgaattctgcaatc

SEQ ID NO: 60 - DNA, 18 bp, Artificial Sequence
  RDS7-P2
ctgaccaactgactgaag

SEQ ID NO: 61 - DNA, 20 bp, Artificial Sequence
  RDS7-F3
gagcgagctgagtggttgtg

SEQ ID NO: 62 - DNA, 21 bp, Artificial Sequence
  RDS7-R3
agtcagttggtcagccatgct

SEQ ID NO: 63 - DNA, 14 bp, Artificial Sequence
  RDS7-P3
cgtctcggaaaccg

SEQ ID NO: 64 - DNA, 17 bp, Artificial Sequence
  RDS8-F1
cgctccgtggccttagc

SEQ ID NO: 65 - DNA, 24 bp, Artificial Sequence
  RDS8-R1
aatctttggagtacgctggatagc

SEQ ID NO: 66 - DNA, 16 bp, Artificial Sequence
  RDS8-P1
cgctactctctctttc

SEQ ID NO: 67 - DNA, 20 bp, Artificial Sequence
  RDS8-F2
gtatgcctgccgtgtgaacc

SEQ ID NO: 68 - DNA, 22 bp, Artificial Sequence
  RDS8-R2
ggcatcttcaaacctccatgat

SEQ ID NO: 69 - DNA, 19 bp, Artificial Sequence
  RDS8-P2
agtgggatcgagacatgta

SEQ ID NO: 70 - DNA, 19 bp, Artificial Sequence
  RDS8-F3
ctgccgtgtgaaccatgtg

SEQ ID NO: 71 - DNA, 23 bp, Artificial Sequence
  RDS8-R3
gcttacatgtctcgatcccactt

SEQ ID NO: 72 - DNA, 17 bp, Artificial Sequence
  RDS8-P3
ctttgtcacagcccaag

SEQ ID NO: 73 - DNA, 24 bp, Artificial Sequence
  RDS9-F1
caggagccaagagtgaagaacagt

SEQ ID NO: 74 - DNA, 25 bp, Artificial Sequence
  RDS9-R1
tggaaaacccaacttctgtacaact

SEQ ID NO: 75 - DNA, 14 bp, Artificial Sequence
  RDS9-P1
tggcagcaagaaaa

SEQ ID NO: 76 - DNA, 26 bp, Artificial Sequence
  RDS9-F2
taacaggtgctaaagtcagagcagaa

SEQ ID NO: 77 - DNA, 23 bp, Artificial Sequence
  RDS9-R2
tcgtcttcctgaatccctttaga

SEQ ID NO: 78 - DNA, 19 bp, Artificial Sequence
  RDS9-P2
agcatttgaaaacatctac

SEQ ID NO: 79 - DNA, 22 bp, Artificial Sequence
  RDS9-F3
cgaatataatcccaagcggttt

SEQ ID NO: 80 - DNA, 19 bp, Artificial Sequence
  RDS9-R3
ccgtggttcgtggctctct

SEQ ID NO: 81 - DNA, 14 bp, Artificial Sequence
  RDS9-P3
ctgcggtaatcatg

SEQ ID NO: 82 - DNA, 27 bp, Artificial Sequence
  RDS9-F4
cacagtgaatcttggttgtaaacttga

SEQ ID NO: 83 - DNA, 22 bp, Artificial Sequence
  RDS9-R4
aaaccgcttgggattatattcg

SEQ ID NO: 84 - DNA, 16 bp, Artificial Sequence
  RDS9-P4
ttgcacttcgtgcccg

SEQ ID NO: 85 - DNA, 23 bp, Artificial Sequence
  RDS12-F1
gagtatggagcagaaacgattgc

SEQ ID NO: 86 - DNA, 27 bp, Artificial Sequence
  RDS12-R1
cagacttttctggtactcttcagtgaa

SEQ ID NO: 87 - DNA, 18 bp, Artificial Sequence
  RDS12-P1
ttcaccaggatccacctc

SEQ ID NO: 88 - DNA, 22 bp, Artificial Sequence
  RDS12-F2
gtatggagcagaaacgattgca

SEQ ID NO: 89 - DNA, 27 bp, Artificial Sequence
  RDS12-R2
cagacttttctggtactcttcagtgaa

SEQ ID NO: 90 - DNA, 17 bp, Artificial Sequence
  RDS12-P2
tttcaccaggatccacc

SEQ ID NO: 91 - DNA, 21 bp, Artificial Sequence
  RDS12-F3
tggttggagagctcatttgga

SEQ ID NO: 92 - DNA, 22 bp, Artificial Sequence
  RDS12-R3
actctcgtcggtgactgttcag

SEQ ID NO: 93 - DNA, 16 bp, Artificial Sequence
  RDS12-P3
ttttgccgatttcatg